(19) United States
(12) Patent Application Publication
(10) Pub. No.: US 2012/0009668 A1
(43) Pub. Date: Jan. 12, 2012

(54) Biogas Capture and/or Collection System

(75) Inventor: Jill Hass, Ottawa (CA)

(73) Assignee: Clearford Industries Inc., Kanata Ontario (CA)

(21) Appl. No.: 13/122,320

(22) PCT Filed: Oct. 2, 2008
(86) PCT No.: PCT/CA2008/001691

§ 371 Date: May 2, 2011

Related U.S. Application Data
(60) Provisional application No. 60/977,024, filed on Oct. 2, 2007.

(51) Int. Cl. C12M 1/107; B65D 85/00

(52) U.S. Cl. 435/300.1; 206/6

(57) ABSTRACT

A biogas capture and/or collection system is provided and comprises one or more biogas capture and/or collection units (BCCU) that capture and/or collect biogas generated in one or more biogas generating chambers (BGC). The BCCUs maybe tubular conduits operatively linked to the BGCs or canisters removably linked with the BGCs and designed for reversible capture of the biogas generated therein. A waste input system operatively linked to the BGCs is used to feed waste from one or more sources of waste thereto. Biogas generation within the BGCs is optionally promoted by retention of at least part of the waste within for a time period sufficient for release of gases due to degradation or by using means for promoting microbial processing such as heating means, centers for applications not limited to electricity production, use as fuels and use for chemical synthesis. The gas utilization centers maybe located either locally at the individual sources of waste or at a centralized location.

### FIELD OF THE INVENTION

The present invention relates in general to the field of biogas generation and in particular to a biogas capture and/or collection system.

### BACKGROUND

Decomposition of waste resulting in generation of biogas occurs as naturally occurring micro-organisms break down and digest the organic waste. In an aerobic system using free gaseous oxygen (or air), the end products of organic waste degradation are primarily CO₂ and H₂O. In an anaerobic system, the intermediate end products of the waste degradation are primarily alchohols, aldehydes and organic acids plus CO₂. In the presence of specialized microbes called methanogens, these intermediates are converted to the final end products of CH₄, CO₂ with trace levels of H₂S.

The formation of methane by methanogens in an anaerobic environment is called methanogenesis.

A simplified overall chemical equation for anaerobic digestion is given below

C₆H₁₂O₆→3CO₂+3CH₄

Methanogenesis has also been shown to use carbon from other organic compounds such as formic acid, methanol, methylamines, dimethyl sulfide, and methanethiol.

The principal products of anaerobic waste digestion are biogas, water and an anaerobic digestate, which can be used as a soil improving material. Biogas is a gaseous mixture comprising mostly methane and carbon dioxide, but also containing small traces of hydrogen and toxic H₂S (formed by the decomposition of the sulphates). The biogas obtained may require further treatment with scrubbing and cleaning equipment (such as amine gas treating) to bring the H₂S levels within acceptable levels and to reduced the quantity of siloxanes (that causes mineralized deposits on the gas engines, resulting in increased wear and tear). Methane obtained from the process can be used for a variety of applications including electricity production and chemical synthesis of compounds including methanol, etc.

Prior systems for biogas generation were not designed to efficiently capture the released gases from the sludge breakdown in decentralized wastewater systems.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a biogas capture and/or a collection system.

In accordance with one aspect of the present invention there is provided a biogas capture and/or collection system comprising one or more biogas capture and/or collection units configured so as to capture and/or collect substantially all of the biogas generated in one or more biogas generating chambers by the degradation of waste received from one or more sources of waste and wherein the sewage treatment center is optionally a leach field.

### BRIEF DESCRIPTION OF THE FIGURES

FIG. 1A-C show different views of a clarifier tank with two compartments operatively linked to a biogas capture and/or collection unit, in accordance with one embodiment of the invention.

FIG. 2 shows one embodiment of the biogas capture and/or collection system where the biogas generating chamber is a sewage holding tank.

FIG. 3 shows one embodiment of the biogas capture and/or collection system where the biogas generating chamber is a septic tank for sewage handling.

FIG. 4 shows one embodiment of the biogas capture and/or collection system where the biogas generating chamber is a clarifier tank in a high-performance sewer system (HPSS).

FIG. 5A shows one embodiment of the biogas capture and/or collection system where a gas utilization center is locally located at the source of sewage.

FIG. 5B shows one embodiment of the biogas capture and/or collection system where the gas utilization center is centrally located and shared by multiple biogas generating chambers.

FIG. 6 shows one embodiment of the biogas capture and/or collection system where the biogas generating chambers are clarifier tanks interfaced to a historic sewer system (HSS).

FIG. 7A shows one embodiment of the biogas capture and/or collection system where a source of sewage feeds into multiple biogas generating chambers.

FIG. 7B shows one embodiment of the biogas capture and/or collection system where one or more sources of sewage feed into a single biogas generating chamber.

FIGS. 8A and 8B show one embodiment of attachment assemblies used to attach the waste input system and waste output system to the biogas generating chamber.

FIGS. 9A-C show the side views of different embodiments of conduits used for fluid communication between the different compartments of a clarifier tank used as biogas generating chamber.

FIGS. 10A and 10B show a flow control mechanism used with the waste output system of a biogas generating chamber, according to one embodiment of the invention.

FIG. 11A shows an end cross-sectional view of the sewage collection mains, in accordance with one embodiment of the invention.

FIG. 11B is a schematic of section of insulated and heated pipe according to one embodiment of the invention.

FIGS. 12-14 show means for heating, aeration, and electrolysis respectively, used for promoting microbial processing within the biogas generating chambers.

FIG. 15A shows an overall layout of a high performance sewer system (HPSS) based on clarifier tanks in one embodiment of the invention. FIG. 15B shows the details of a clarifier tank used in FIG. 15A. The biogas capture and/or collection units of the clarifier tanks are not shown for the sake of clarity.

FIGS. 16A & 16B show the side and plan perspective views of a soil filter and vent for use with a HPSS, according to one embodiment of the invention.

FIGS. 17A & 17B show the side and plan perspective views of a manhole and cleaning system for use with a HPSS, according to one embodiment of the invention.

FIGS. 18A & 18B show the plan and side view of a pumping station to be used with a HPSS, according to one embodiment of the invention.

FIG. 19 is a side view of an electrode assembly according to one embodiment of the invention.

FIG. 20 shows a layout of a HPSS for a residential community, according to one embodiment of the invention, and shows the connection of the gas collection mains and sewage collection mains to the multiple clarifier tanks.

FIG. 21 shows the side view of a HPSS trench containing both the gas and sewage collection mains, in accordance with one embodiment of the invention.

FIG. 22 shows the side view of a HPSS layout wherein both the gas and sewage collection mains are contained in the same trench beneath the center of the road, in accordance with one embodiment of the invention.

FIG. 23 shows side view of a clarifier tank according to one embodiment of the invention. The sewage inlet and outlet pipes used for waste in/out put are also shown.

FIGS. 24A to 24E show different views of a clarifier tank according to one embodiment of the invention.

FIG. 25A shows one embodiment of a biogas generating chamber: a clarifier tank comprising two compartments in series. FIGS. 25B shows another embodiment of a clarifier tank comprising more than two compartments in a series.

FIG. 26A shows one embodiment of a clarifier tank comprising multiple compartments that form a combination of parallel and serial paths for sewage handling.

FIGS. 26B-D show cross-sections of three embodiments of clarifier tanks with hybrid parallel and serial paths for sewage handling.

FIG. 27A-B show two embodiments of clarifier tanks with three compartments differing in the geometrical arrangement of the compartments wherein FIG. 27B shows a tank with a stepped floor.

FIG. 28 shows one embodiment of a clarifier tank with three access ports and two compartments in fluid communication with each other.

FIG. 29 shows one embodiment of the system comprising on-site biogas harnessing for sludge reduction comprising a compact gas compression, flare and heating system which includes a sludge blanket heating system or coil.

FIG. 30 shows one embodiment of the system comprising a methane mitigation means comprising a soil vent attached to the biogas collection pipe via a gooseneck pipe.

FIG. 31 shows a side view of a clarifier tank according to one embodiment of the invention. The sewage inlet and outlet pipes used for waste in/out put are also shown. A methane capture compartment with associated methane collection pipe is also shown.

### DETAILED DESCRIPTION OF THE INVENTION

### Definitions

As used herein, the term ‘about’ refers to a +/−10% variation from the nominal value. It is to be understood that such a variation is always included in any given value provided herein, whether or not it is specifically referred to.

The term “waste” is used to define the solid, liquid, and gaseous substances that enter the biogas generating chamber. Examples of suitable ‘waste’ include but are not limited to municipal wastes; wastes produced by industrial activity; sewage and manure.

The term “sewage” as used herein, include but is not limited to agricultural wastes, residential sewage, biomass and industrial sewage.

The term “liquid effluent” is used to define the substantially liquid portion of the sewage.

The term “sludge” is used to define the substantially solid portion of the sewage.

The term “microbe” is used to include bacteria and other micro-organisms.

Unless defined otherwise, all technical and scientific terms used herein have the same meaning as commonly understood by one of ordinary skill in the art to which this invention belongs.

The invention comprises a system for the capture and/or collection of biogas generated from waste such as sewage. The system comprises one or more Biogas Capture and/or Collection Units (BCCU) for use with one or more Biogas Generating Chambers (BGC) operatively linked therewith, for the capture and/or collection of the biogas generated therein.

The BCCUs can be interconnected to each other, for example, using a system of pipes. Alternatively, the BCCUs can function as stand-alone units that are harvested on an appropriate periodic basis for the biogas stored therein. The stored biogas can optionally be used on-site for a variety of applications including domestic applications.

The biogas extracted using the BCCUs may be utilized in gas utilization centers that may be located either locally at the individual sources of waste or at a centralized location. The biogas is optionally used at the gas utilization centers for one or more of a variety of applications including but not limited to electricity production, use as fuels and use for chemical synthesis. Alternatively, the biogas can be flared to convert the methane to carbon dioxide or be used to heat the BGC or converted to carbon dioxide through the action of methanotrophs in a soil vent.

Biogas is produced within the BGCs by degradation of waste received from one or more sources using a waste input system such as a system of pipes. The waste input system could optionally incorporate some pre-treatment stages such as sorting, crushing or focussed pulse technology. The biogas generation may be optionally promoted by retention of at least part of the waste within the BGC for a time period sufficient for release of gases due to degradation or by using means for promoting microbial processing such as heating means, aeration means, or a means for producing in situ oxygen and hydrogen.

Biogas Capture and/or Collection Units

The biogas capture and/or collection system comprises one or more Biogas Capture and/or Collection Units (BCCU) for use with one or more Biogas Generating Chambers (BGC) operatively linked therewith, for the capture and/or collection of the biogas generated therein. Optionally, the BCCUs are structured such that they create minimal disruption to the operation of the BGC and configured so as to remove a substantial portion of the gases generated therein.

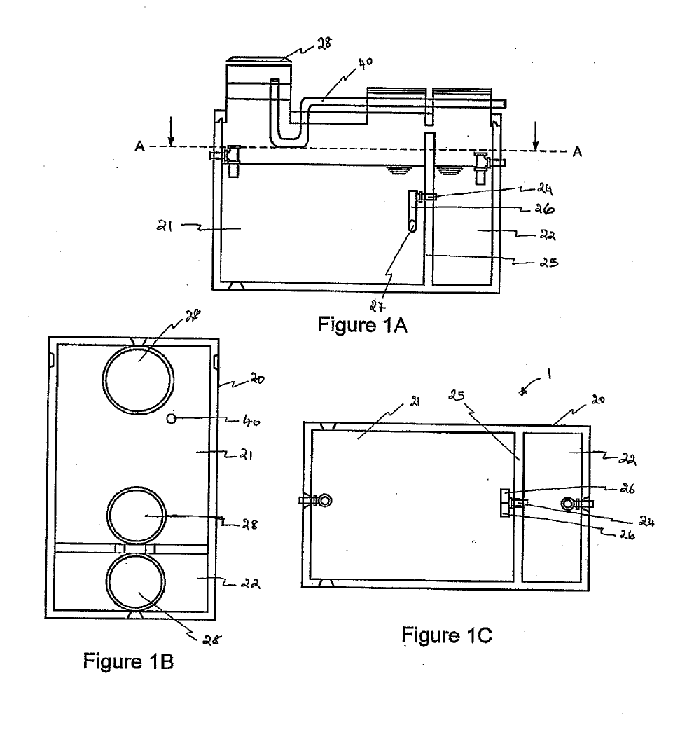

In one embodiment of the invention and referring to FIG. 1A, wherein the BGCs are adapted for use as clarifier tanks in a sewage handing system, the BCCUs are intended to maximize the capture of biogas from the BGC and is optionally placed near the top of the first compartment of the BGC. This position takes into account two factors: (a) biogas generation occurs mostly in the first compartment where the sludge is predominantly collected and undergoes degradation; (b) biogas is lighter than air and therefore tends to collect near the top of the BGC.

In one embodiment of the invention, the biogas streams collected by one or more BCCUs are combined together, for example, using a system of pipes. In one embodiment of the invention, the BCCUs function as stand-alone units that are harvested on an appropriate periodic basis for the biogas stored therein.

The BCCUs can use active means, passive means or some combination thereof for the capture and/or collection of the biogas from the BGCs. In one embodiment of the invention, the BCCU is passive and comprises one or more tubular conduits that are operatively linked to the BGCs for capturing the biogas within. In one embodiment of the invention, the BCCU uses an active suction technique with the tubular conduits to extract the biogas from the BGCs in slugs or with continuous suction.

In one embodiment of the invention, the BCCUs are tubular conduits connected to one or more biogas transfer elements (BTE) for transport of the biogas to one or more gas utilization centers. In one embodiment of the invention, the BCCUs are containers such as canisters removably linked with the BGCs and designed for reversible capture of the biogas generated therein. Optionally, the containers are filled with materials designed for reversible capture of gases of a chosen molecular family.

### Tubular Conduits

In one embodiment of the invention, the BCCUs are conduits attached to the BGC using attachment assemblies. A worker skilled in the art will understand that the different types of attachment assemblies as are known in the art are intended to be included within the scope of this invention.

In one embodiment of the invention and referring to FIG. 1A, the BCCUs are U-shaped conduits so that any non-gaseous substance that inadvertently enters the BCCU (e.g. during overflow of the BGC) is not transferred further downstream. The BCCU is positioned and structured so as to exit the BGC through a slab on top of the BGC, thus preserving the integrity of the BGC, as the BCCU does not exit the BGC on its sides.

In one embodiment of the invention, the conduit acting as the BCCU is made of HDPE. The flexible nature of HDPE reduces the chances of shearing damage to the pipe. HDPE is also non-corrosive to the typical gases extracted from sewage. Sealing means as are known to a worker skilled in the art such as mentioned earlier can be used to seal the connection between the BGC and the BCCUs.

Optionally, the connection of the BCCUs to the BGC is made using a sealingly airtight connection. Substantial air-tightness of all connections in the sewer system can be tested on site in a manner similar to that of testing the integrity of septic tanks, i.e., a vacuum test, which would be known to a worker skilled in the art. The portion of the sewer is sealed, a vacuum is applied and periodic readings with a gauge are used to determine whether the section is losing its vacuum.

### Reversible Capture Units

In one embodiment of the invention, the BCCU is a container such as a canister removably attached to the BGCs and designed for reversible capture of the biogas generated therein. In one embodiment, the BCCUs are a hybrid combination of conduits and canisters, wherein conduits operatively linked to the BGCs captures the biogas generated therein and transports it to removably attached canisters that reversibly capture the biogas.

On saturation with captured biogas, the canister or its contents therein is dissociated from the BGC and optionally transported to a facility (e.g. the gas utilization center) where the biogas captured is extracted again for further processing, storage and/or utilization. A canister-based biogas collection method is well suited for stand-alone septic systems and holding tanks where the absence of a sewage collection main avoids the need for trenches.

A variety of materials can be used within the canisters for capturing the biogas either using adsorption or other mechanisms. Some of these materials are described below. A worker skilled in the art will understand that the materials listed below are merely exemplary and other materials suitable for capture of gases as are known in the art are also to be construed as included within the scope of the invention described herein.

In one embodiment of the invention, the biogas is collected in canisters packed with adsorbent materials. The biogas, comprising primarily of methane, is adsorbed in the pores and on the surfaces of the adsorbent medium. Methane molecules preferentially adsorb in pores having a diameter of 1.0-1.5 nm. In one embodiment of the invention, the canister is filled with a material that has a high volume of pores less than 1.6 nm in width as a percentage of total pore volume are used.

Activated carbon has long been used for removal of impurities and recovery of useful substances from liquids and gases because of its high adsorptive capacity, wherein “activation” refers to any of the various processes by which the pore structure is enhanced. In one embodiment of the invention, highly microporous carbon is used within the canisters for capturing the biogas. The microporous carbon can be prepared by a variety of different techniques such as further chemical activation of activated carbon. An example of a process for preparation of highly microporous carbon is given in U.S. Pat. No. 5,626,637.

The container can also be filled with materials whose lattice structures of crystalline or grain configuration is capable of reversibly trapping the methane molecules. In one embodiment of the invention, these materials have lattice structures that permit the penetration of methane molecules to the interior of the solid mass and have an inner surface activity with respect to the methane molecule such as to allow surface adhesion at least to the extent necessary to augment the trapping effect. In one embodiment of the invention, zeolites of known cage-like lattice structure, such as mentioned in U.S. Pat. No. 4,495,900 are used.

In one embodiment of the invention, the container can be filled with a sulphur-containing active carbon, produced from inexpensive aromatic precursors, such as chrysene, coal tar, and petroleum oils. An example of a process for producing such a material is given in U.S. Pat. No. 5,639,707.

In one embodiment of the invention, the BCCU canisters are filled with nanoporous carbon made from waste corn cob. In this embodiment, corn cobs are baked into carbon briquettes that trap biogas in fractal pore spaces. The fractal nature of the pores results in higher capture efficiency than other structures. The pore size affects the biogas collection capability of the carbon briquettes. Based on the type of activation procedures, about 80 different types of carbon can be produced from corn cob.

In one embodiment of the invention, biogas is collected from the BGC by promoting the formation of clathrate hydrates. Clathrate hydrates are a class of solids in which gas molecules occupy “cages” made up of hydrogen-bonded water molecules. These cages are unstable when empty, collapsing into conventional ice crystal structure_{;} but they are stabilized by the inclusion of appropriately sized molecules within them. Most low molecular weight gases such as O₂, H₂, N₂, CO₂, H₂S, Ar, Kr, Xe and methane, as well as some higher hydrocarbons and freons will form hydrates under certain pressure-temperature conditions. Once formed, clathrates can usually be decomposed by increasing the temperature and/or decreasing the pressure.

The extracted biogas can be utilized for a variety of applications at one or more gas utilization centers. In one embodiment of the invention, methane is separated out from the extracted biogas at a centralized gas utilization center and is used for electricity production. In one embodiment of the invention, the gas utilization centers are located locally at the individual sources of waste. In one embodiment of the invention, the gas utilization center is centralized and shared by multiple BGCs.

### Biogas Generating Chambers

The biogas generating chamber (BGC) is a closed container that receives waste from one or more sources of waste via a waste input system. Biogases are generated within the BGC by the degradation of waste. One or more biogas capture and/or collection units (BCCU) are operatively linked to the BGCs for extraction of the biogas generated therein. Optionally, a waste output system is operatively linked to the BGC for removal of at least part of the waste therefrom. A worker skilled in the art will understand that biogas generation within the BGC can be promoted using a variety of different techniques.

The BGC can be made of concrete, such as high strength, reinforced concrete of at least 35 mPa (4,500 psi), but may also use any suitable material such as fibre-glass, high density polyethylene (HDPE), or other materials known to a worker skilled in the art that would allow for the desired level of system sealing.

The BGC can be constructed in a variety of shapes. The dimensions of the BGC are determined based on its application of use. A skilled worker will appreciate that the dimensions of the BGC are chosen to accommodate the application for which it is used. In one embodiment of the invention, the BGC is used to receive sewage from a single residence and has a volume range between 3,600-4,500 liters. A BGC used to receive sewage from a multi-residence building or industrial waste may have a higher volume.

Optionally, the BGC comprises two or more compartments wherein the adjacent compartments are separated by interior walls. FIGS. 1A and 1B show the top view and side view of a BGC with two compartments 21 and 22, in accordance with one embodiment of the invention. FIG. 1C shows a top sectional view of the embodiment. Optionally the upper edge of the interior wall 25 is slightly lower than the upper edge of the BGC 20 to allow for gas exchange between the two compartments 21, 22. The interior wall 25 also comprises a conduit 24 that allows operative communication between the two compartments. The dimensions of the compartments of the BGC are chosen based on the application requirements. The BGC optionally has one or more openings and lids 28 on the top to enable access for maintenance, repairs and other purposes that will be readily known to a worker skilled in the art.

Referring to FIG. 31, in one embodiment, the first compartment of the BGC is equipped with a methane capture compartment or screen which functions to localize the methane to the methane collection pipe. FIG. 31 also shows various other features including an air flow control flow valve and wastewater venting through pipe.

In one embodiment of the invention, the waste is sewage. The BGC can be used as a sewage holding tank, a septic tank, or a clarifier tank. As a clarifier tank, the BGC can be used either as part of a high-performance sewer system (HPSS) such as described below, or interfaced to a historic sewer system (HSS).

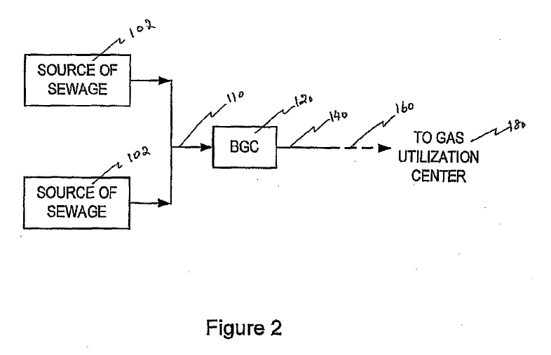

In one embodiment of the invention and in accordance with FIG. 2, the BGC 120 is a sewage holding tank. A waste input system 110 is used to transport sewage from one or more sources of sewage 102 into the BGC 120 wherein gases are generated due to the degradation of the sewage. In one embodiment of the invention, the waste input system 110 comprises one or more sewage inlet pipes. The gases released from the BGC 120 are extracted using one or more BCCUs 140 and sent to gas utilization centers 180 for a variety of applications as described above. In this application, the BGC 120 is usually sealed off and abandoned when it is substantially filled.

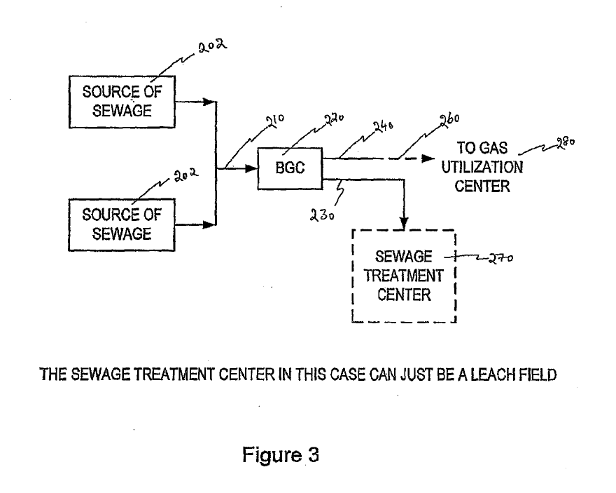

In one embodiment of the invention and referring to FIG. 3, the BGC 220 is a septic tank. Sewage enters through the waste input system 210 into the BGC 220 wherein the sludge settles to the bottom. In one embodiment of the invention, the waste input system 210 comprises one or more sewage inlet pipes. The substantially liquid portion of the sewage, i.e., the liquid effluents, is then drained off using a waste output system 230 to a leach field where the remaining impurities decompose in the soil and the water is eliminated through percolation into the soil. In one embodiment of the invention, the waste output system 230 comprises one or more sewage outlet pipes. Optionally, the BGC 220 comprises two compartments, the first one of which receives sewage from the source(s) of sewage 202 and allows most of the sludge to settle, while the second one allows for any additional settlement of the sludge and for the outlet of the liquid effluents to the leach field. The sludge is frequently removed from the BGC 220 to ensure efficient operation. One or more BCCUs 240 are used to extract the gases released due to sludge degradation within the BGC 220.

In one embodiment of the invention and referring to FIGS. 1 & 25A-B, the BGC 20 is a clarifier tank used for handling sewage. The BGC 20 comprises a cascade of two or more compartments 21, 22, 23 in fluid communication with each other using conduits 24. The first compartment 21 is used to receive sewage from one or more sources of sewage through one or more sewage inlet pipes serving as the waste input system 10. Sludge settles in the first compartment 21 while the liquid effluent flows from the first compartment, through conduits 24 into the second compartment 22. The second compartment 22 allows any remaining sludge particles suspended in the liquid effluent to settle out before the liquid effluent passes on to the remaining compartments 23. Provision of additional compartments 23 permits additional sludge particles to settle from the liquid effluent before discharge therefrom. One or more BCCUs 40 extract the gases released due to the degradation of sludge within the various compartments 21, 22, 23 of the BGC 20. When the one or more access hatches 28 are secured to the BGC 20 and the sewage inlet 10 and outlet pipes are plugged, the BGC 20 is substantially airtight.

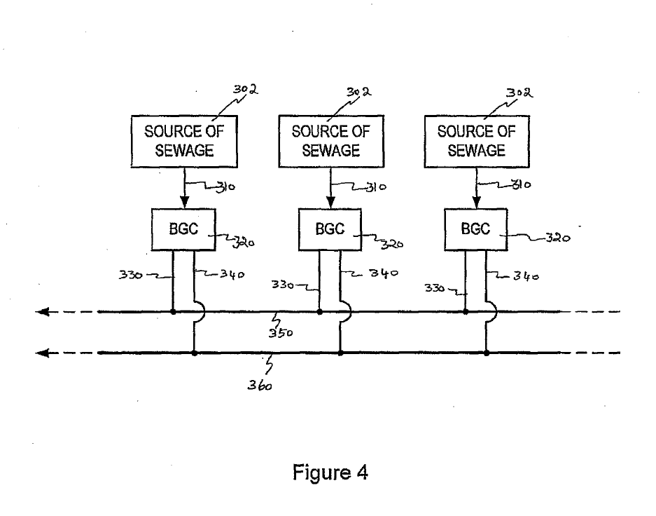

In one embodiment of the invention and referring to FIG. 4, the BGC 320 is a clarifier tank used as part of a high performance sewer system (HPSS) 301. HPSS is particularly well adapted to be installed in remote areas and areas with large amounts of rock near the ground surface that impedes the use of private sewage disposal systems such as septic systems. Here, the BGCs 320 collect sewage from sources of sewage 302 such as residences and carry the liquid effluent using sewage collection mains 350 to a sewage treatment center (not shown in FIG. 4) for processing.

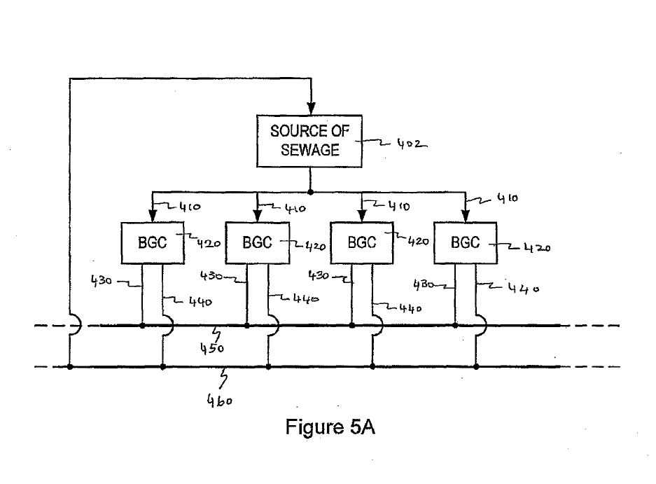

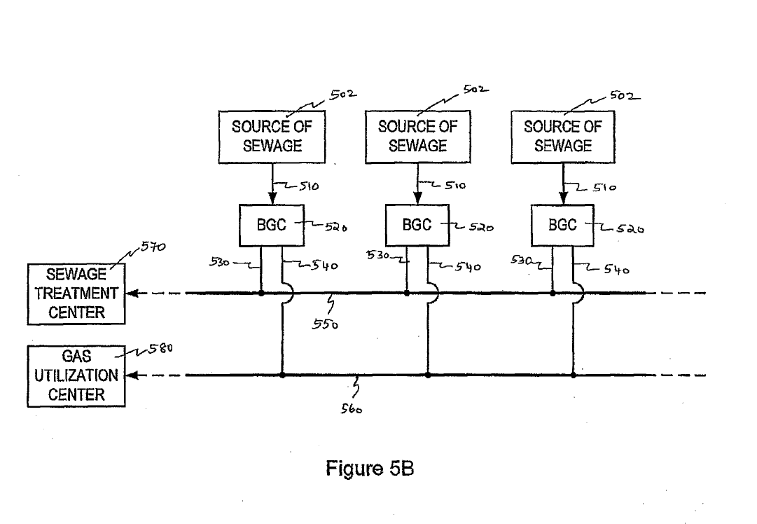

In one embodiment of the invention 501 and referring to FIG. 5B, the sewage treatment center 570 is centralized and is shared by multiple BGCs 520. By separating the sludge substantially in each compartment, the liquid effluent in the sewage collection main 350, 550 that is received from the last compartment of the BGC 320, 520 is effectively pre-treated before it enters the sewage treatment center 570. This can result in a reduction in size and complexity of a centralized sewage treatment center 570. Additionally, any sludge that precipitates from the liquid effluent in the latter compartments may also degrade and be removed similar to that of the first compartment.

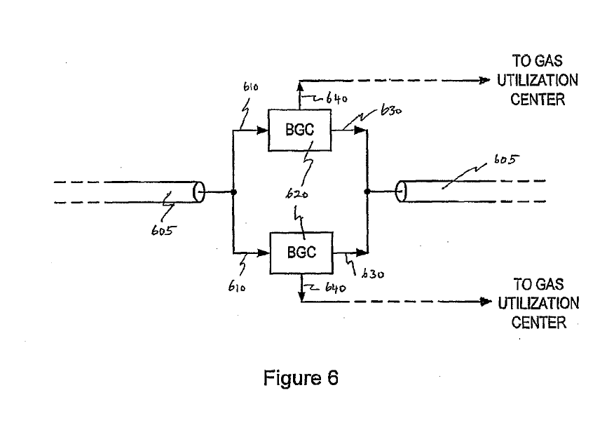

In one embodiment of the invention 601, an existing HSS 605 is retrofitted to interface with clarifier tanks used as BGCs 620. With reference to FIG. 6, the existing HSS 605 is redirected to one or more BGCs 620 using the waste input system 610. For efficiency, the BGCs 620 for this application are typically larger than those installed at individual residences in an HPSS. Once settling of the sludge has occurred in the first compartment of the BGCs 620, the liquid effluent is conducted back to the HSS 605 through the sewage outlet pipes that serve as the waste output system 630. This embodiment allows communities to draw the benefits of clarifier tanks without replacing their existing HSS.

In accordance with one embodiment of the invention and referring to FIGS. 1A-C, the BGC may optionally have one or more lids 28 and openings 29 on the top that can be used for maintenance, repairs and access. In one embodiment of the invention, they are also used for removal of the sludge. Installation of at least one lid flush with the ground level enables easy access for routine maintenance and sludge removal without disruption to the surrounding land. Additional elements may be added to the openings to prevent unauthorized or accidental entry into the BGC after installation.

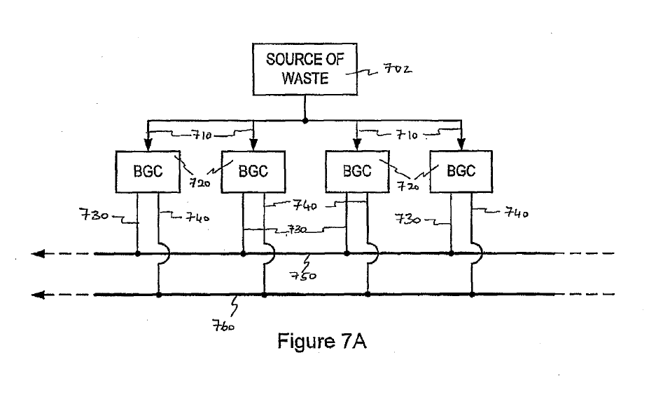

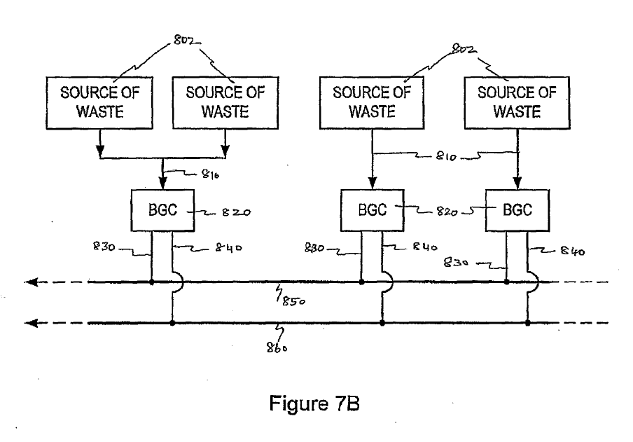

In each of the above scenarios, each source of sewage may be connected to one or several BGCs or several sources of sewage may be connected to one BGC, depending upon the sewer demand and land availability. FIGS. 6 and 7 show the connection of one source of sewage 702 to multiple BGCs 720 and the connection of multiple sources of sewage 802 to one BGC 820 respectively.

Biogas generation can be promoted within the BGCs using a variety of techniques. A key factor in biogas generation is the provision of sufficient time for breakdown of waste. The amount of biogas generated increases as the time for waste breakdown increases. Biogas generation can also be promoted by optimizing environmental conditions, such as temperature, pH, components, nutrient levels, moisture or water-content and aeration levels.

### Promotion of Biogas Generation by Increase Time for Sewage Breakdown

The time available for breakdown of the waste is highly dependant on the design of the overall waste management system. In one embodiment of the invention, the time for sewage breakdown can be increased by proper design of the BGCs and/or the overall sewage processing system.

In embodiments of the invention where the BGC comprises of two or more compartments and is used for handling sewage, the sludge portion of the sewage received from one or more sources of sewage undergoes settling in the various (predominantly in the first) compartments of the BGC while the liquid effluent flows out of the BGC to an HSS, HPSS, or a leach field (in the case of a septic tank) using one or more sewage outlet pipes. As only the sludge remains in the BGC, cleanout cycles can be long. In one embodiment of the invention, the first compartment is connected to a siphon such that sludge can be extracted from the BGC during routine cleanout.

The sludge settling to the bottom of the first compartment of the BGC is reduced by the action of microbial digestion. Larger first compartments that retain a larger volume of sludge extend cleanout cycles; act as surge suppressors to slow the flow of sewage through the system; and increase the hydraulic retention time. All these factors result in enhanced settling of the sludge in the first compartment and thus enhanced biogas generation.

Over time, three substantially distinguishable sewage layers develop in the first compartment of the BGC: 1) the scum layer, which is substantially liquid and sludge. The scum is composed of materials that have a lower specific gravity than water, such as grease, oil, and fats: 2) the middle layer comprises liquid and suspended solids, wherein these solids are typically very small organic materials that continue to be degraded while in the liquid layer; 3) the bottom sludge layer contains materials that have a higher specific gravity than water, are denser than water and are derived from much of the solid sludge.

A worker skilled in the art will understand that depending on whether the BGCs are connected to a HSS, HPSS or a leach field, the various components of the system including but not limited to the vents, pipes, joints of pipes to other components, conduits, pumping stations etc. will have differing design requirements.

### Promotion of Biogas Generation by Optimization of Environmental Conditions

In one embodiment, biogas generation can be promoted by optimizing environmental conditions, such as temperature, pH, components, nutrient levels, moisture or water-content and aeration levels. In one embodiment of the invention, the. BGC comprises a means for optimizing one or more environmental conditions to promote microbial digestion. Optionally, the BGC can further comprise a means for monitoring environmental conditions within the solid portion of the waste including one or more sensors, for example without limitation, temperature sensors, pH sensors, moisture sensors, aeration sensors and the like. In one embodiment of the invention, the BGC comprises a feedback system responsive to environmental cues as a means for optimizing one or more environmental conditions in response to signals received from one or more sensors.

### Control of Temperature

In one embodiment of the invention, the rate of microbial digestion of sludge in the BGC is optimized through the addition of heat. Maintaining the temperature of the sludge within an optimal range can increase the rate of digestion. Increasing the temperature inside the BGC optimizes the growth rate of the micro-organisms that break down the sludge. A worker skilled in the art would be aware of the optimal temperature range required for efficient microbial reactions.

For example, depending on the methanogens species present, there are two conventional temperature ranges of operation for anaerobic digestion: (a) Mesophilic: takes place optimally around 37-41° C. or at ambient temperatures around 25-45° C. with mesophiles as the digestion agents; and (b) Thermophilic: takes place optimally around 50-52° C. at elevated temperatures up to 70° C. with thermophiles as the digestion agents.

Mesophilic bacteria are more tolerant to changes in environmental conditions than the thermophiles. Therefore, mesophilic digestion systems are considered to be more stable than thermophilic digestion systems. However, the latter facilitate faster reaction rates and hence faster gas yields at increased temperatures.

In one embodiment of the invention, there is provided a BGC that is insulated to increase and/or maintain a constant desired optimum temperature with reference to the ambient temperature outside of the BGC which may or may not be optimal.

In one embodiment of the invention in which the BGC is located partially or fully above ground, at least part of the BGC is painted black or manufactured from material that absorbs solar heat.

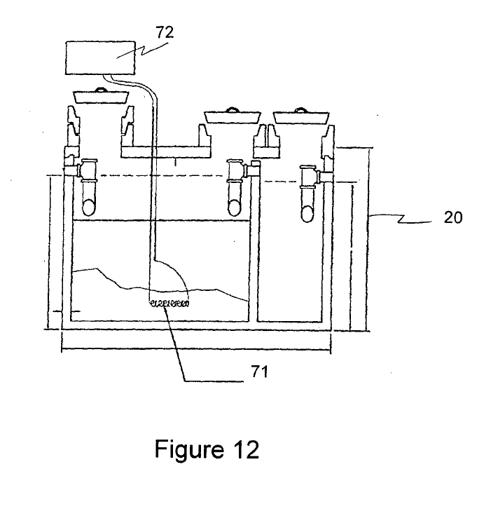

With reference to FIG. 12, in one embodiment of the invention, the temperature in the BGC is increased through a heating means 71. The heating means can be powered by a power source 72 such as a solar panel array, or other source as would be readily understood by a worker skilled in the art. The heating means can either be located within the BGC or external to the BGC. Optionally, the heating means can be powered by captured biogas.

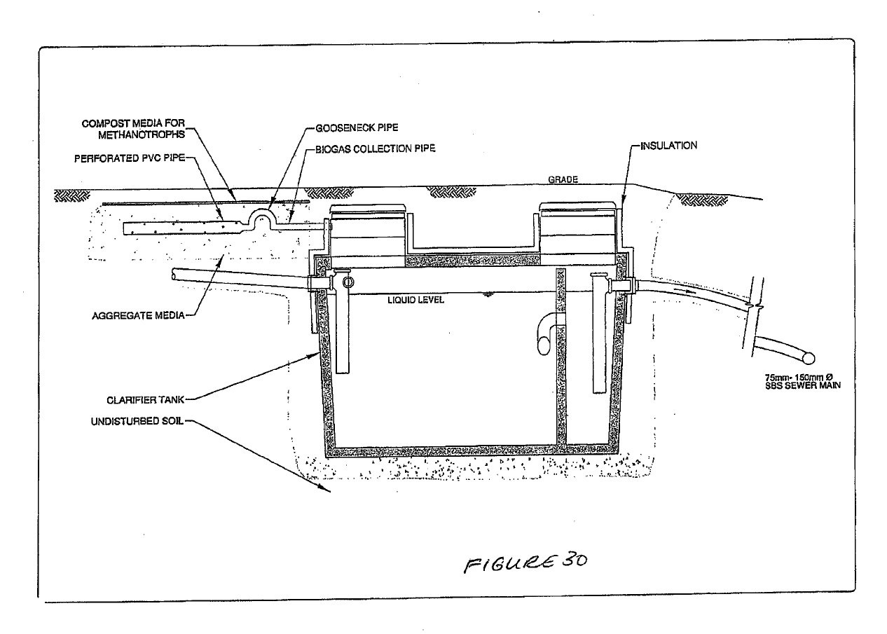

With reference to FIG. 29, the system may further comprise an on-site methane harnessing system for sludge reduction comprising a gas compression flare and heating system and sludge blanket heating system. Such an on-site methane harnessing system provides for the chemical conversion of methane gas into carbon dioxide by flaring gas on-site and supplying the heat produced to the sludge blanket to expedite the sludge degradation process and extension of the pump out cycle.

In embodiments in which heating means are external to the BGC, the heating means include means for heating the walls of the BGC such as slab heaters. Alternatively, waste containing a solid component can be pre-heated prior entering a BGC.

In one embodiment, the heating means also comprises a temperature sensing means such as a thermostat. In one embodiment, the heating means also comprises a feedback system which receives information from a temperature sensor, such as a thermostat, and controls the heating means so as to maintain a preset optimal temperature.

### Control of Aeration

Increasing oxygen available to microbes promotes aerobic digestion of the waste within the BGC while limiting oxygen promotes the production of methane-containing biogas by anaerobic digestion. By creating localized zones within the BGC that promote either aerobic digestion or anaerobic digestion, methane containing biogas production can be maximized and sludge accumulation minimized. Effective aeration can be accomplished by either pre-settling aeration of the waste such that anaerobic zones are established as oxygen is utilized or by post-settling aeration of the waste in a location specific manner. Aeration can be provided either through the introduction of air or high-purity oxygen and may be intermittent or continuous.

In one embodiment of the invention, the level of aeration will be within a range that maintains the biomass' energy requirements and supports efficient facultative bacterial reactions without contributing to the net production of new biomass.

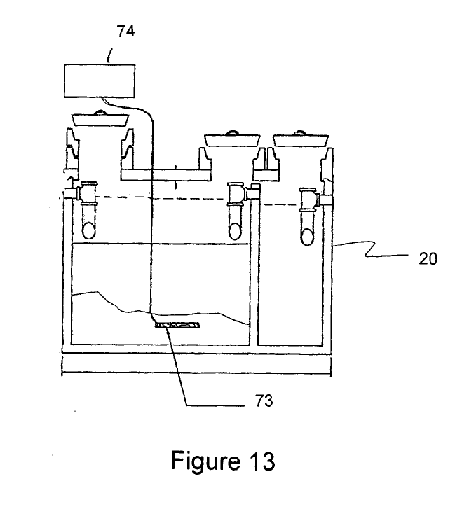

With reference to FIG. 13, in one embodiment of the invention, there is provided aeration means comprising a compressor 74 that pressurizes air and delivers it into the BGC; and a diffuser 73 that distributes the air inside the BGC 20 to allow the sludge to be broken down through aerobic digestion. Means for diffusion are known in the art and include coarse bubble diffusers, fine bubble diffusers, jet aerators, static aerators, and mechanical mixers or mechanical surface aerators, or other aeration devices as would be readily understood by a worker skilled in the art. The compressor system can be powered by a power source (not shown) such as a solar panel array, or other power source as would be readily understood by a worker skilled in the art.

### Control of Production of In Situ Oxygen And Hydrogen

The in situ production of oxygen and hydrogen stimulates both aerobic and anaerobic processing. The oxygen is used as an electron acceptor by the aerobic bacteria, while the hydrogen is consumed in anaerobic reactions and can stimulate the digestion process beyond the acidogenesis phase to methanogenesis.

Means for the in situ generation of oxygen and/or hydrogen are known in the art and can include any mechanism capable of electrolysis, including one or more electrolytic cartridges, cells or chambers. In one embodiment of the invention, the mechanism capable of electrolysis is capable of water electrolysis. In one embodiment of the present invention, the mechanism capable of electrolysis is capable of generating oxidizing agents.

The type of water electrolysis apparatus that are appropriate for use in the instant invention will vary according to the functional requirements for the system. A worker skilled in the art will appreciate that the electrolysis apparatus can function intermittently or continuously. The electrolysis apparatus can be turned on or off either in a pre-programmed manner or in response to signals, e.g. from sensors.

In one embodiment, the electrolysis apparatus comprises two or more electrodes and an energy or power source.

In one embodiment, the electrodes are located within the sludge layer.

In one embodiment, the electrolysis apparatus comprises a process controller operatively connected to one or more electrolysis apparatus and one or more sensors. The process controller can comprise a device capable of receiving and interpreting signals from the one or more sensors, processing the received signals and sending commands to one or more electrolysis apparatus to optimize results with substantially minimum energy costs. The process controller can also perform supervisory functions, such as monitoring for system failures, etc.

In one embodiment, the process controller further comprises a sensing means for detecting pH levels and, in order to prevent acidification of the sludge due to H+ build up, enabling the electrolysis of water to be regulated in a pH-dependent manner.

### Electrolysis Apparatus

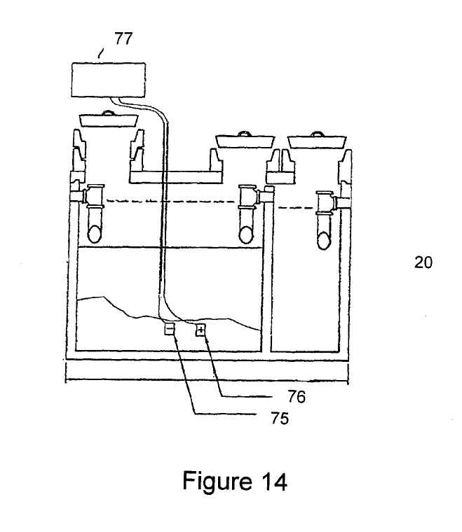

In one embodiment of the invention, the electrolysis apparatus comprises two or more electrodes located on the inner surface of the BGC. With reference to FIG. 14, in one embodiment of the invention, two electrodes 75 and 76 are operatively connected to a power source 77, located externally to the BGC 20. During water electrolysis, the cathode 75 or negative electrode generates hydrogen and the anode 76 or positive electrode generates oxygen. Alternatively, the electrolysis unit may generate other (non-oxygen) oxidizing agents.

By promoting the digestion of the accumulated sludge within the: BGC, the electrolysis apparatus indirectly serves to increase the cleanout periods. The accumulation of sludge for longer periods serves to enhance the biogas generation.

There are various types of electrodes known in the art, including flat screen, mesh, rod, hollow cylinder, plate, or multiple plates, among others. A worker skilled in the art would know which type of electrode is appropriate for use in the instant invention according to the functional requirements of the system.

Solid particles adhere to bubbles that rise to the surface and out of the treatment zone. In addition, when oxygen bubbles form, inefficiencies in the system are created as oxygen fails to properly diffuse. In one embodiment of the invention, the configuration of the anode will be selected to reduce or prevent the formation of gas bubbles.

The electrode may be composed of a variety of materials. The electrode material must be sufficiently robust to withstand the elevated voltage and current levels applied during the electrolytic process of the invention, without excessive degradation of the electrode. A given electrode may be metallic or non-metallic. Where the electrode is metallic, the electrode may include platinized titanium, among other compositions, as would be readily understood by a worker skilled in the art. Where the electrode is non-metallic, the electrode may include graphitic carbon, or can be one or more of a variety of conductive ceramic materials, as would be readily understood by a skilled worker.

The anode and cathode of the electrode cell may have a variety of different compositions and/or configurations without departing from the scope of the invention.

In one embodiment of the invention, the anode and cathode are substantially equivalent in order to facilitate bipolar operation to reduce scale build-up on the electrodes. Electrolytic processes may generate thin films or deposits on the electrode surfaces that can lower the efficiency of the water treatment process. De-scaling of the electrodes to remove some films may be carried out by periodically reversing the polarity of operation (switching the anode and cathode plates to the opposite polarity). Automatic logic controls permit programmed or continuous de-scaling, thus reducing labour and maintenance costs.

In one embodiment of the invention, a reference electrode is integrated into the electrolysis apparatus. A reference electrode is an electrode that has a well-known and stable equilibrium electrode potential that is used as a reference point against which the potential of other electrodes may be measured. While a variety of electrode configuration can fulfill the above requirements, a suitable reference electrode for the invention would be readily understood by a worker skilled in the art and can include silver/silver-chloride electrode, calomel electrode, and a normal hydrogen electrode, among others.

In one embodiment of the invention, at least one of the one or more electrodes is substantially submerged in the sludge. In one embodiment, all of the electrodes are substantially submerged in the sludge. In one embodiment of the invention, at least one of the one or more electrodes is partially submerged in the sludge. In one embodiment, all of the electrodes are partially submerged in the sludge.

The placement of the electrodes will vary based on the system requirements. The electrodes may be in a fixed position or movably mounted. The electrodes may be mounted on the walls and/or floor of the BGC. In one embodiment of the invention, the electrodes are suspended within the sludge using means known in the art.

Appropriate energy sources for the electrolysis apparatus are known in the art and the skilled technician will know which energy source is most appropriate for configuration of the system. The energy source will deliver a controlled electrical charge having a value determined by the requirements of the system. The energy or power source may be a standard or rechargeable battery, direct AC connection or solar power, amongst others known in the art.

### Other Processes

The process of micro-aeration generally relates to the optimization of environmental conditions within the sludge such that microbial processing is facilitated.

In one embodiment of the process, the sludge is heated or aerated.

In one embodiment of the invention, the pH of the sludge or components thereof is adjusted to alter microbial processing.

In one embodiment of the invention, the microbial population is adjusted either by changing conditions or by seeding sludge with specific microbes.

In one embodiment of the invention, the sludge is sterilized prior to seeding, for example by heat or ozone treatment.

In one embodiment of the invention, oxygen and hydrogen are generated in situ intermittently or continuously by water electrolysis.

In one embodiment of the invention, other oxidizing agents are generated in-situ.

Integration with Other Solid Waste Reduction Systems and Methods

The system and processes described above for substantially optimizing solid waste decomposition can be integrated with other systems and processes for minimizing solid waste including, for example, pre- or post-enzymatic treatment, and others.

In one embodiment of the invention, the system and processes of the invention are integrated with systems for pre-treating sewage using electrolysis, for example as disclosed in U.S. Pat. Nos. 4,089,761 and 4,124,481.

A worker skilled in the art will readily understand that one or more of the systems for promoting microbial processing as described herein can be combined.

### Biogas Delivery to the Gas Utilization Centers

The biogas extracted using the BCCUs is optionally utilized in gas utilization centers for one or more of a variety of applications including but not limited to electricity production, use as fuels and use for chemical synthesis. In one embodiment of the invention and referring to FIG. 5A, the gas utilization centers are located on-site at the sources of waste. In one embodiment of the invention and referring to FIG. 5B, the gas utilization center is a centralized facility shared by multiple BGCs.

In one embodiment of the invention, the biogas generated in the BGCs is captured using containers designed and configured to reversibly capture the biogas, that serve as BCCUs. These containers are then moved to gas utilization centers where they are treated to release the captured biogas (‘desorption’) therein. A worker skilled in the art will understand that the methods for desorption vary with the type of material used in the canisters and that all such methods are to be considered within the scope of this invention. The desorption process can either be done immediately on receipt of the containers, or till such time as the biogas is to be utilized in which case the containers serve as storage devices.

Alternatively, the containers can be moved to intermediate locations where they undergo desorption and the extracted biogas is then transported to the gas utilization centers using Biogas Transport Elements (BTE), such as a system of pipes.

In one embodiment of the invention, the biogas is collected using BCCUs in the form of tubular conduits which are connected to one or more BTEs, such as a system of pipes, to gas utilization centers for further processing, storage and/or utilization. In the case of a centralized gas utilization center, the BTE serves as a gas collection main.

In one embodiment of the invention, the BTEs are made using flexible, pressure-rated high density polyethylene (HDPE) pipe, typically between 19-100 mm in diameter. The use of this type of pipe offers many of the advantages such as ease of installation, fewer joints between pipe sections, reduction of open excavation and surface reinstatement etc. The BTEs can also be made from a variety of other materials such as polyethylene. The use of HDPE ensures that the remains uncorroded for a design period of greater than 100 years.

In one embodiment of the invention where the gas utilization center is centralized and the BGCs are adapted for use in a HPSS, the BTE is placed in the same trench as the sewage collection mains. The use of the same trench for both the sewage collection mains and the BTE results in significant cost savings. Other services may also be added in the same trench, thus, providing “bundled services”.

A worker skilled in the art will understand that precautions will have to be taken to ensure that there is no biogas leak to the environment either from the. BTEs or at the gas utilization centers. This includes the user of butt-welding or other connection sealing method known to the skilled worker in this art to ensure that all the connections and joints are sealingly connected. The substantial air-tightness of connections between sections of BTE can be verified on site using a vacuum test as discussed above. The methane produced in the BGCs may be mixed with trace gases to instil a noticeable pungent smell that can be used to detect any methane leaks. Gases that can be used for this include but is not limited to butyl mercapton.

The BTEs may also comprise standard gas flow equipment such as pressure monitors, valves, compressors etc inserted to control the flow of gases. A worker skilled in the art will readily understand the appropriate placement of these devices along the BTEs. In one embodiment of the invention, the gas flow equipment serve to ensure a uniform pressure for the extracted gas flow. In one embodiment of the invention, these flow control devices are controlled to either operate the gas extraction process intermittently or continuously. Typical flow control mechanisms for gases such as pressure valves can be used, as will be readily understood by a worker skilled in the art.

In embodiments of the invention where predominantly methane is extracted from the BGC, it is important to ensure that there is minimal in/ex filtration into/from the BTE as the mixing of methane with air can result in a flammable mixture at concentrations of methane between 5% and 15%. Security measures may be placed within the BTEs and at the gas utilization centers to ensure that there are no explosions or unwanted leaks. These security measures include but are not limited to pressure sensors.

### Biogas Processing & Applications

In one embodiment of the invention, filtering means are used to remove or isolate specific gases. For example, these filtering means can be used to isolate methane. :A worker skilled in the art will readily understand that these filtering means can be placed anywhere in the path of the gas flow including but not limited to the following locations: within the BGC, within the BCCUs, within the BTEs, or at the gas utilization centers.

Other post-processing steps may be applied to the biogas streams collected by the BCCUs. In one embodiment of the invention, scrubbing techniques maybe applied to remove. H₂S from the biogas stream. A worker skilled in the art will readily understand that other post-processing steps as are known in the art are understood to be within the scope of the invention.

In one embodiment of the invention, predominantly methane is collected from the BGCs and transported using the BTEs to a centralized plant either for industrial use in chemical synthesis or for the production of electricity. In one embodiment of the invention, the methane is used for electricity generation by burning it as a fuel in gas turbines, steam boilers, reciprocating engines or micro-turbines. Compared to other hydrocarbon fuels, burning methane produces less. CO₂ for each unit of heat released, and also produces the most heat per unit mass.

In one embodiment of the invention, the methane collected can be transported as fuel in liquefied form similar to liquid natural gas (LNG). Methane in the form of compressed natural gas (CNG) is also used as a fuel for vehicles and is considered to be more eco-friendly than gasoline and diesel.

Methane is also used as a feedstock for the production of hydrogen, methanol, acetic acid and acetic anhydride in the chemical industry. A worker skilled in the art will readily understand the different design issues associated with the handling of methane in the context of different downstream applications. In one embodiment of the invention, the methane collected from each BGC is pumped back upstream for applications such as electricity production for the residences.

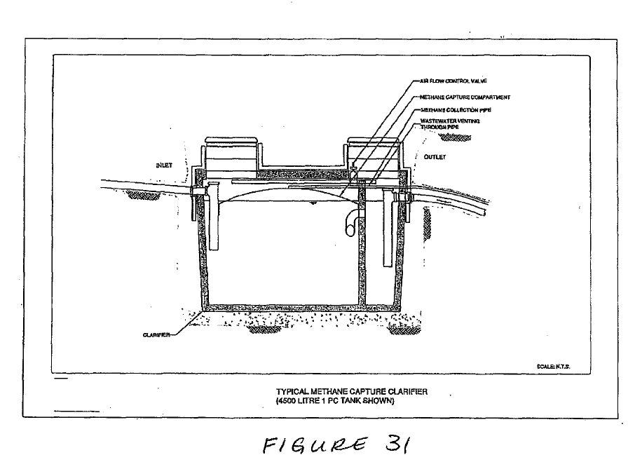

In one embodiment, the captured methane is converted on-site to carbon dioxide. Referring to FIG. 30, the system may be equipped with a methane mitigation means that promotes the biological conversion of methane gas into carbon dioxide by methanotrophic microbes which thrive in certain soils and compost media. The methane. mitigation means comprises a soil vent and a subsurface trench with media to encourage the growth of methanotrophs. Optionally, the solid vent comprising a perforated PVC pipe is connected to the biogas collection pipe via a gooseneck pipe. The soil vent is housed within a subsurface trench that includes media such as compost media that supports methanotroph growth.

In one embodiment, the methane mitigation means comprises a methane abatement means. Methane abatement means are known in the art and include catalytic converters. Optionally, heat generated by the catalytic converter during the conversion of methane is used to heat the sludge blanket.

### EXAMPLE 1

Here, we describe a biogas capture and/or collection system adapted for use with a high-performance sewer system (HPSS) based on clarifier tanks that serve as BGCs. The system is designed so that the flow of liquid is predominantly driven by gravity, while assisted by pumps at key locations. The HPSS is designed to collect sewage from a source of sewage such as a residence and carry the liquid effluent to a central sewage treatment center for processing. The HPSS is particularly well adapted for installation in remote areas and areas with large amounts of rock near the surface that impedes the use of private sewage disposal systems.

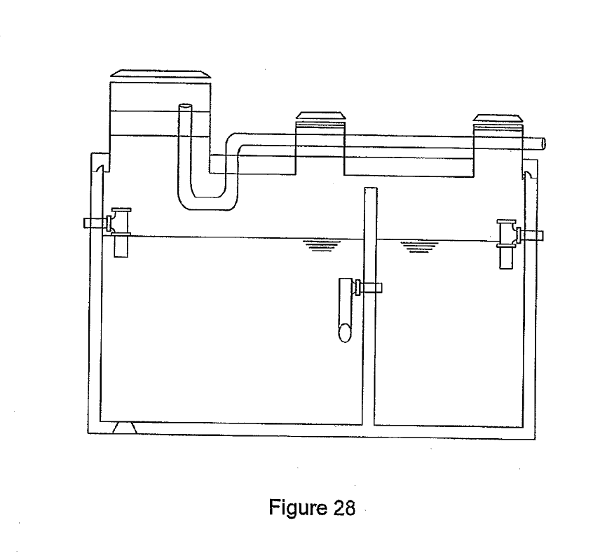

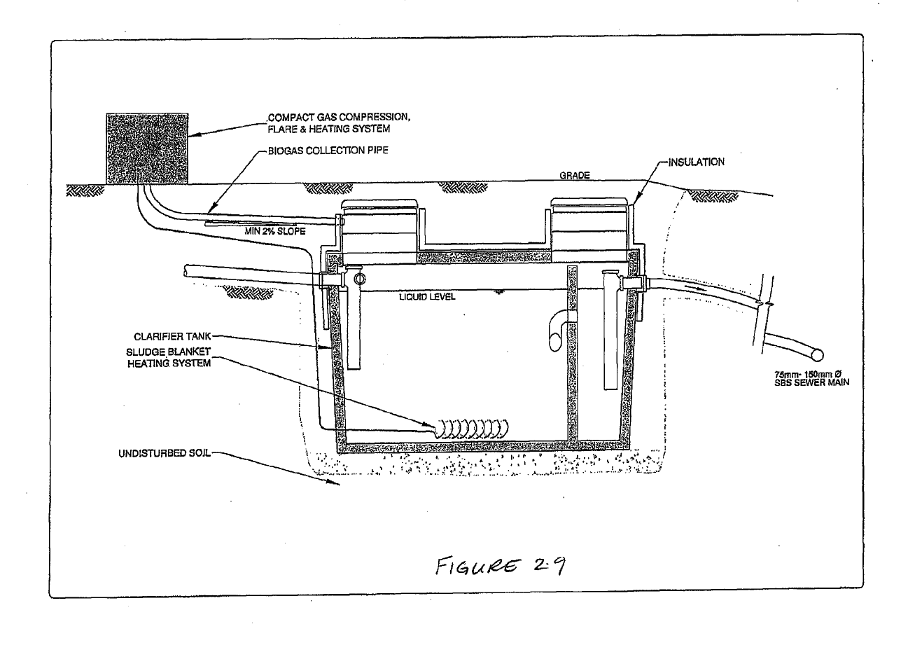

Referring to FIGS. 28A-C, the sewage inlet pipe 12 brings sewage from a source of sewage 2 to the BGC 20, which comprises a first compartment 21 and a second compartment 22, separated by an interior wall 25. The upper edge of the interior wall 25 is slightly lower than the upper edge of the BGC 20 which allows gas exchange between the two compartments 21 and 22.

The sewage is transferred to the first compartment 21 of the BGC where the sludge 3 substantially settles, while the liquid effluent 4 flows through a conduit 24, into the second compartment 22. The second compartment 22 allows any remaining sludge 3 particles suspended in the liquid effluent 4 to settle out. Additional compartments if present, allow for further separation of the sludge 3 from the liquid effluent 4. Thus, the liquid effluent 4 eventually leaving the BGC 20 through a sewage outlet pipe 32 is effectively pre-treated.

Over time, there are substantially three distinguishable sewage layers which develop in the first compartment 21 of the BGC 20: 1) the scum layer, which is substantially liquid and sludge. The scum is composed of materials with a lower specific gravity than water such as grease, oil, and fats: 2) the middle layer comprises liquid and suspended solids, wherein these solids are typically very small organic materials and continue to be degraded while in the liquid layer; 3) the bottom sludge layer contains materials that have a higher specific gravity than water, are denser than water and are derived from much of the solid portion of sewage waste.

The sludge settling within the BGC accumulates for a period of time when it is reduced by the microbial action resulting in biogas generation. The reduced sludge is removed periodically using a siphon operationally connected to the BGC 20 during routine cleanout. Typically, for a BGC used in a residential application, the first compartment 21 can handle up to 17 years of accumulated sludge, although a 7-10 year cleanout maintenance cycle can enable the system to operate within a desired efficiency level.

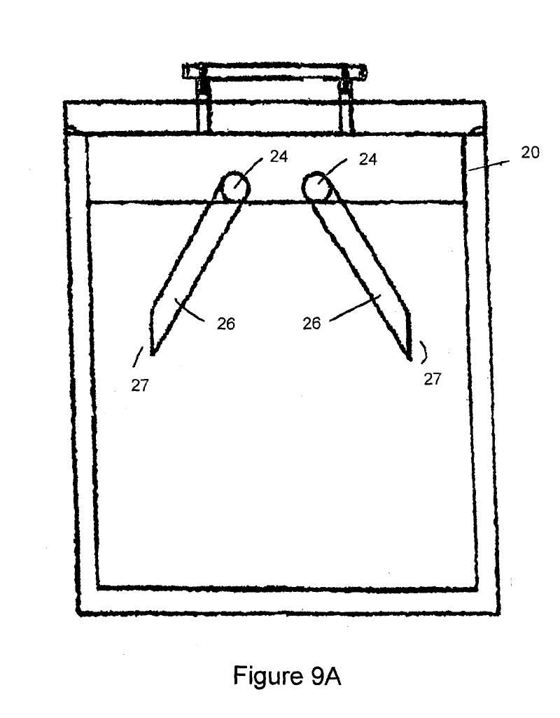

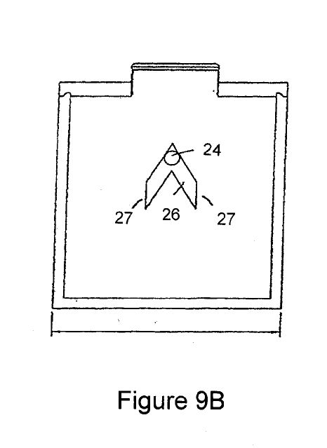

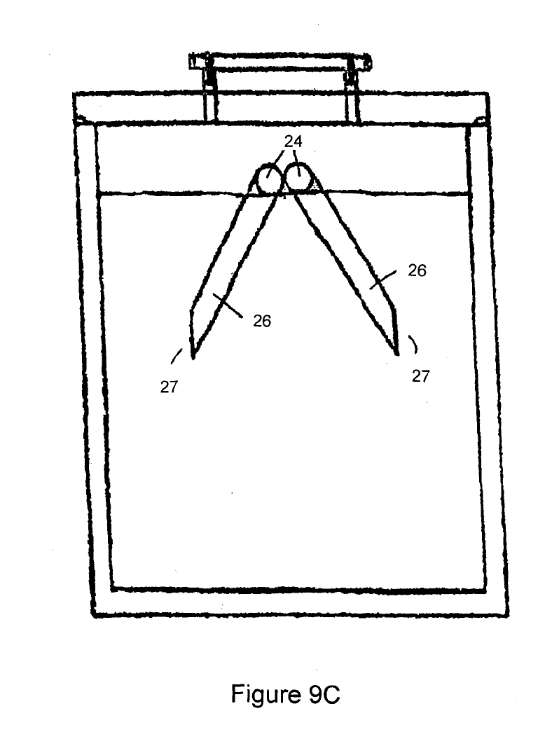

Referring to FIGS. 1A and 9B, the conduit 24 is located in the first compartment 21 adjacent the interior wall 25 and is positioned such that the opening 27 is below the scum layer and above the sludge layer. One or more hollow tubes 26 extend from the conduit vertically downwards towards the bottom of the BGC. The one or more tubes 26 are positioned during manufacturing at an angle of at least 60 degrees relative to the vertical axis of the BGC 20. This reduces the TSS levels in the liquid sewage leaving the BGC by preventing particular matter attached to gas bubbles from entering the conduit 24.

The BCCU used for biogas collection is a U-shaped conduit 40 made of HDPE and placed near the top of the first compartment 21 of the BGC 20. The U-shape ensures that any liquid that might enter the BCCU is not transferred further downstream. The BCCU is positioned and structured so as to exit the BGC 20 through a slab on top of the BGC. The position of the BCCU near, the top of the first compartment of the BGC ensures that biogas collection is maximized. The flexible nature of HDPE reduces the chances of shearing damage to the pipe. HDPE is also non-corrosive to the typical gases extracted from sewage. Sealing means is used to seal the connection between the BGC and the BCCUs.

Referring to FIGS. 1A and 1B, the BGC 20 comprises one or more openings 29 and lids 28 in its top to enable easy access to the compartments 21, 22 of the BGC 20 for maintenance and repairs as well as removal of sludge 3. At least one lid 28 is positioned such that it can be removed to gain access to the first compartment 21. The openings 29 are of sufficient diameter to allow for any crust formed by the hardening of the oily scum layer at the top, to be broken up and removed in order that the sludge 3 can then be efficiently removed. At least one lid 28 is installed such that it is flush with the ground level when the BGC 20 is installed to provide easy access for routine maintenance and solid sewage removal without disruption to the surrounding land. With reference to FIG. 1A, rings are connected to an opening 29 in the BGC 20 to bring the lid 28 flush with the ground. Rings or risers can be made of PVC or any other type of material as would be known to a worker skilled in the art which would enable rings to be easily and sealably connected to the BGC 20 at the time of installation.

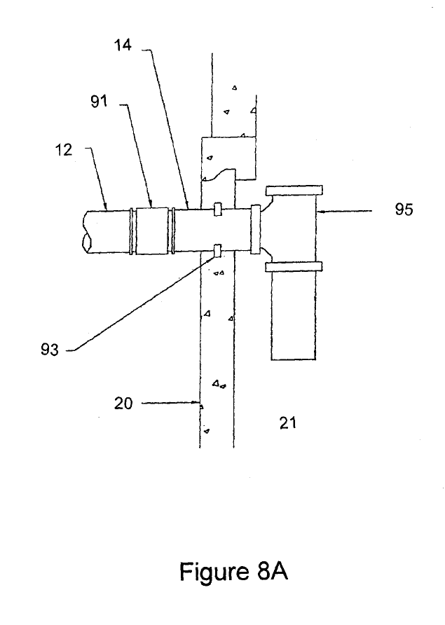

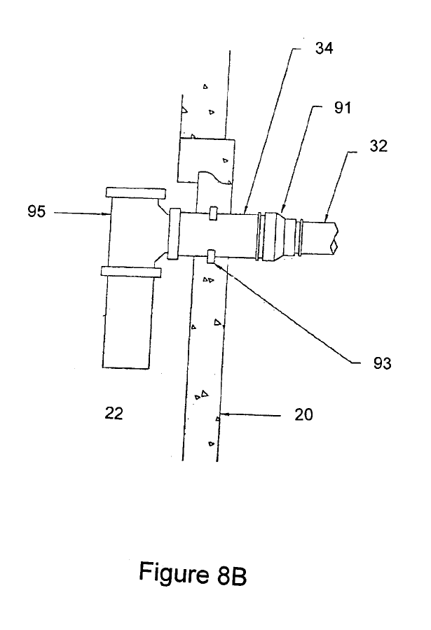

With reference to FIGS. 1A, 1C, 8A and 8B, the sewage inlet pipes 12 and sewage outlet pipes 32 are attached to the BGC 20 through an attachment assembly. Referring to FIGS. 8A and 8B, the attachment assembly comprises a collar 91; one or more substantially airtight gaskets 93, inlet pipe 14 or outlet pipe 34 and one or more tee pipes 95. The collar 91 fits into the inlet or outlet pipe 14 or 34, which extends through and beyond the side of the BGC 20. Located on the inside of the BGC 20 are one or more tee pipes 95 which connect to the collar 91 through the inlet or outlet pipe 14 or 34. The seal between the inlet 14 or outlet pipe 34 and the BGC 20 can be made substantially airtight by the utilization of one or more A-LOK gaskets 93. With regard to FIG. 8B, the diameter of lateral sewer pipe 32 is less than the diameter of outlet pipe 34. A bell shaped connector 92 is used to connect the two pipes 32 and 34 together. The outlet pipe 34, and the collar 91, or bell shaped connector 92, are heat welded or, by use of another suitable method, fused with pipe 12 or 32. Substantial airtightness of all connections in the sewer system can be tested on site in a manner similar to that of testing the integrity of septic or clarifier tanks, i.e., a vacuum test, which would be known to a worker skilled in the art. The portion of the sewer is sealed, a vacuum is applied and periodic readings with a gauge are used to determine whether the section is losing its vacuum. Results can be achieved immediately.

The sewage inlet pipe 12 and sewage outlet pipe 32 are constructed of flexible HDPE. The use of flexible HDPE pipe at the inlet and outlet points of the. BGC prevents shearing that might otherwise occur as the BGC or pipe settles or shifts in the ground following installation thereof. A worker skilled in the art would be aware of normal ranges of differential movement based on the individual soil conditions present at installation and would provide sufficient slack in the inlet and outlet pipes to compensate for such movement.

The entire HPSS is designed such that all connections are air tight. Thus on closing of the vents (described later), the entire system is air tight. However during operation the vents are kept open to avoid hydraulic lock. The sealed configuration of the components and connections provide a means for pressure testing and ensures that no inlexfiltration occurs during operation. All the components are sealed such that the HPSS can be pressure tested when the vents are sealed, and therefore the system is substantially air tight. The BGC 20 is pressure tested and pre-plumbed prior to installation.

Multiple sealing means are provided to seal the connection between the BGC and the sewage inlet and outlet pipes in order to account for excessive differential movement between the pipes and the BGC, due to for example thermal expansion and ground freezing. A worker skilled in the art would be aware of appropriate sealing means necessary to provide a substantially airtight seal, for example without limiting the foregoing, gaskets, flexible membranes and the like.

The sealing means is designed to be sufficiently flexible to compensate for relative movement between the pipes and the BGC in the plane of the wall of the BGC while still maintaining a substantially airtight seal. The sealing means is sufficiently flexible to compensate for relative movement between the inlet and outlet pipes and the BGC perpendicular to the plane of the wall of the BGC while still retaining a substantially airtight seal. This flexibility is necessary to account for thermal expansion coefficient differences between the pipe and the BGC. The difference in expansion coefficients is a factor of the materials from which the pipes and the BGC are constructed; as would be known to a worker skilled in the art.

The BGC comprises one or more flow attenuation devices that moderate flow rates leaving the BGC. The use of flow attenuation devices thus provides a more consistent flow rate of the liquid effluent leaving the BGC, enabling smaller pipe sizes throughout the HPSS, substantially eliminating instantaneous surge loads, and enhancing peak shifting.

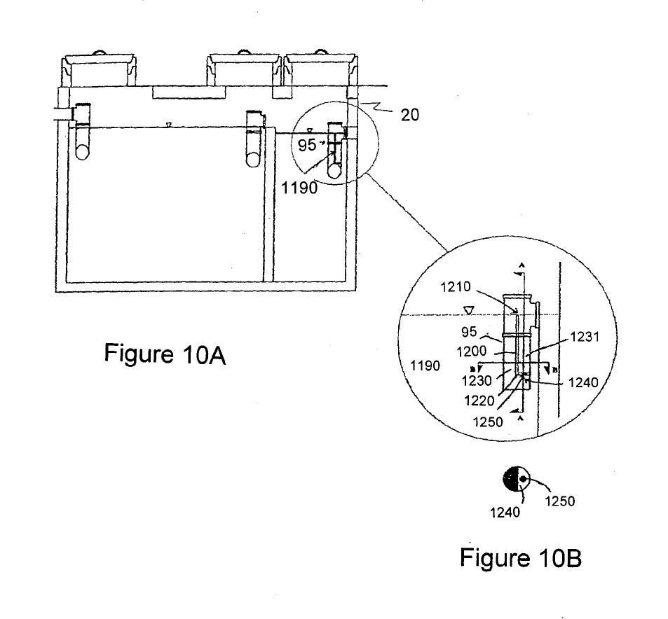

With reference to FIGS. 10A and 10B, the flow attenuation device 1190 is integrated into the outlet tee pipe 95 wherein the interior of the outlet tee pipe 95 comprises one or more partitions 1200 that divide the outlet 95 along its longitudinal axis into two or more sections 1230, 1231. The one or more partitions comprise a top edge 1210 located nearest to the top of the BGC 20 and an opposing lower edge 1220. At least one of said one or more sections 1231 of said tee pipe 95 possesses a plug 1240 that prevents liquid effluent from entering the section 1231. Said plug 1240 comprises one or more orifices 1250 that limit the flow of liquid effluent into the section 1231. As a hydraulic load is placed on the BGC 20, liquid effluent initially has a period of restricted flow through the one or more orifices 1250. The one or more partitions 1200 are designed such that the top edge 1210 of the one or more partitions 1200 is higher than the point at which the tee pipe 95 connects to the outlet pipe 34. The one or more partitions 1200 may be used as an overflow mechanism during sustained high hydraulic loading and the top edge 1210 of the one or more partitions 1200 may be cut horizontally or be equipped with a weir or an equivalent graduated flow mechanism.

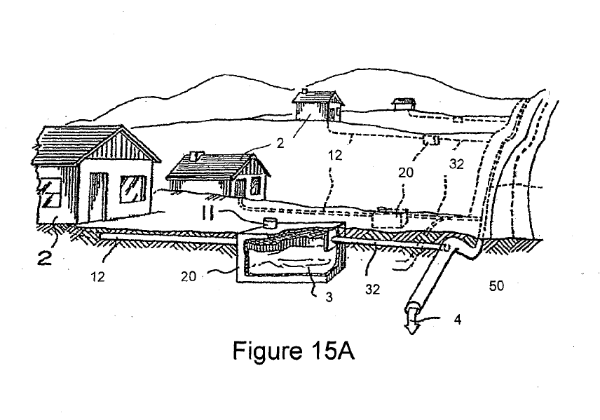

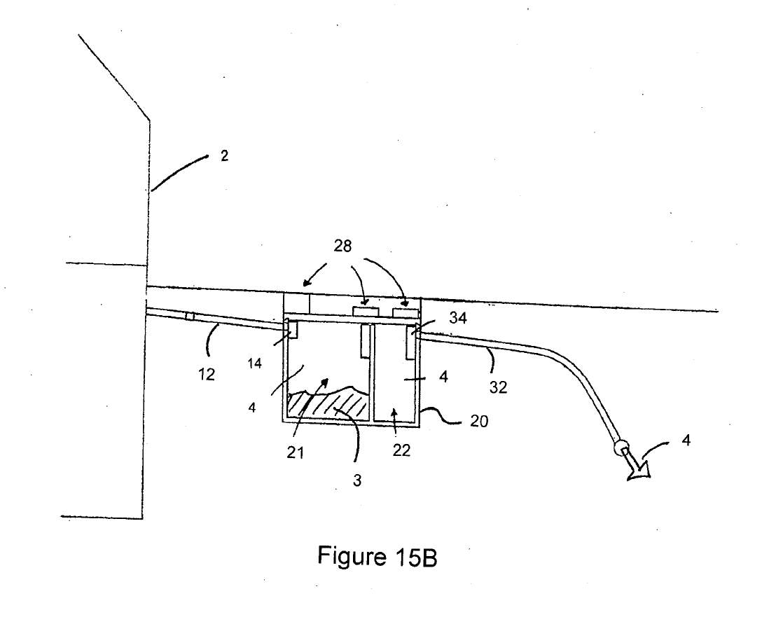

Referring to FIG. 15A, the liquid effluent 4 leaves the BGC 20 through the sewage outlet pipe 32 which carry it to the sewage collection main 50. The sewage, outlet pipe 32 and the sewage collection main 50 are substantially smaller in diameter than those in HSS. Pressure-rated, flexible, high-density. polyethylene (HDPE) pipe, typically between 50-150 mm in diameter, is used as the sewage collection main. Their joints are sealingly formed, for example by heat welding or other technique known to a worker skilled in the art, thereby substantially eliminating any infiltration of groundwater and exfiltration of liquid effluent from the sewer system. The flexibility of the pipe enables the design of the system to take into account the topography and the geology of the land to optimize the flow of liquid through the system. The substantial air-tightness of connections between sections of pipe can be verified on site using a vacuum test as discussed above.

The sewage collection main 50 is designed with non-corrodible components and has a design life of over one hundred years. The system is designed so that post-construction pipe settling does not have an adverse effect on the hydraulic performance of the HPSS.

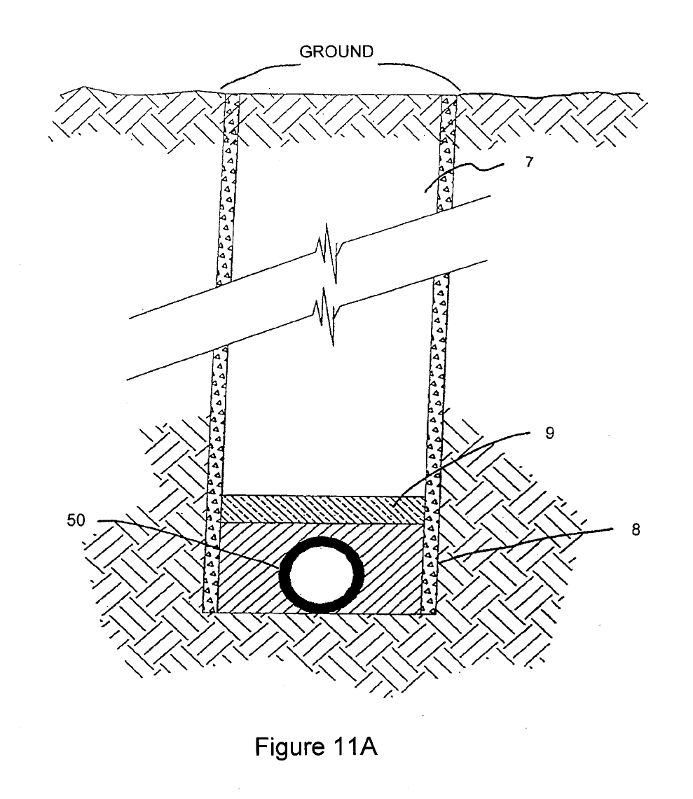

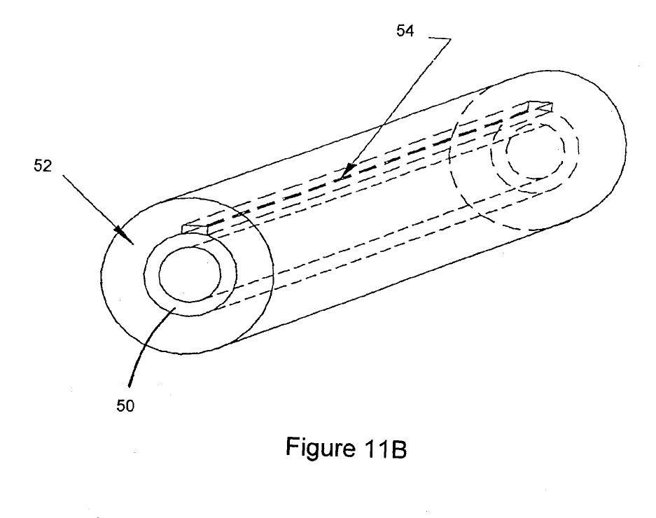

Freezing of the sewage and liquid effluent within the sewage collection mains can result in cracked pipes and blockages: Therefore, referring to FIG. 11A, the sewage collection mains 50 are placed beneath the frost line in a trench 7 resulting in insulation by the surrounding soil. The sewage collection main 50 is surrounded by a sand bedding 8 and covered with insulation material 9 such as Styrofoam.

For extra protection from low temperatures and with reference to FIG. 11B, the sewage collection main 50 is sheathed in insulating material 52, such as Styrofoam. Additionally, one or more heat traces 54 comprising a copper wire operatively connected to one or more heat sources (not shown) is located within the sewage collection mains 50. Heat is conducted through the copper wire and prevents liquid in the mains 50 from freezing.

The installation is typically done alongside or underneath the community roads or boulevards. The trenches are not as wide or deep as in traditional HSS. The trenches can be dug with a backhoe, trencher, or other excavation equipment. Alternatively, the pipes can be installed by horizontal directional drilling. In areas that are predominantly composed of rock, trenches can be made by blasting and removal of the raw material. Horizontal drilling techniques reduce installation time, minimize disruption to residents or local businesses and substantially reduce surface reinstatement costs.

For proper flow of the liquids through any system such as an HPSS or an HSS, without air locks, venting is required, especially in areas of inflection, in areas after turbulent flow or in open flow. Vents are designed to allow gas exchange between the sewer system and the surrounding environment but configured to prevent the escape of sewage or liquid effluent from the system or the inflow of groundwater into the system.

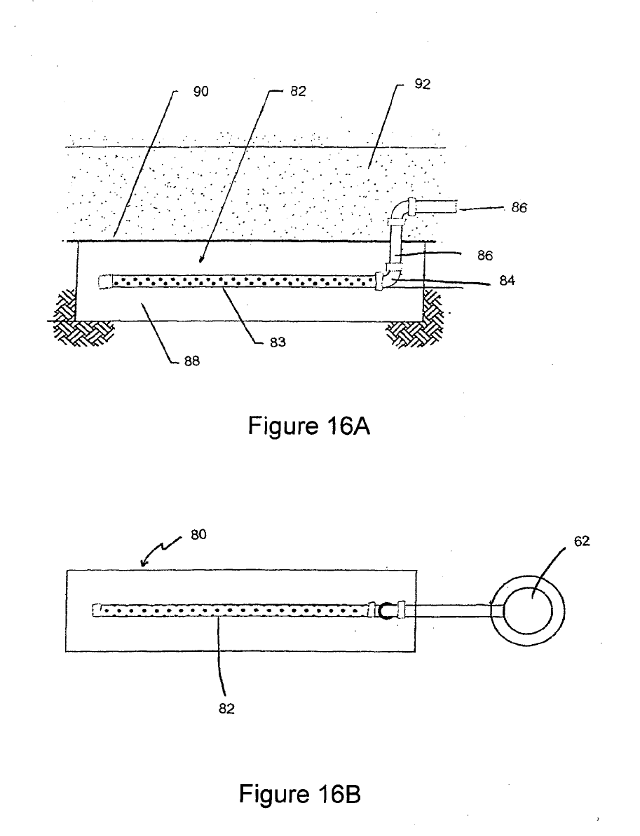

With reference to FIGS. 16A-B, there is provided a lateral vent 82 comprised of a perforated pipe 83 located in a bed of clear stones 88. The vent 82 is connected to the sewer system by means of elbow joints 84 and pipe 86 connected in such a way as to prevent the infiltration of groundwater and the exfiltration of liquid effluent into the surrounding environment. The configuration of elbow joints 84 and pipe 86 necessary to prevent this will depend to some degree on the placement of the vent within the system and would be well known to a worker skilled in the art.

The system further comprises sealed maintenance clean-outs which are provided to accomplish a similar function as maintenance holes or manholes in historic sewer systems. The clean-outs are constructed of suitable material such as high-grade, durable plastic. According to the installation environment and the length of each pipe coil, clean-outs may be installed 100 m to 300 m or more apart, also depending on the venting requirements. The clean-outs provide easy access for routine flushing, which may occur every 7-10 years, after desludging of the upstream primary processing units.

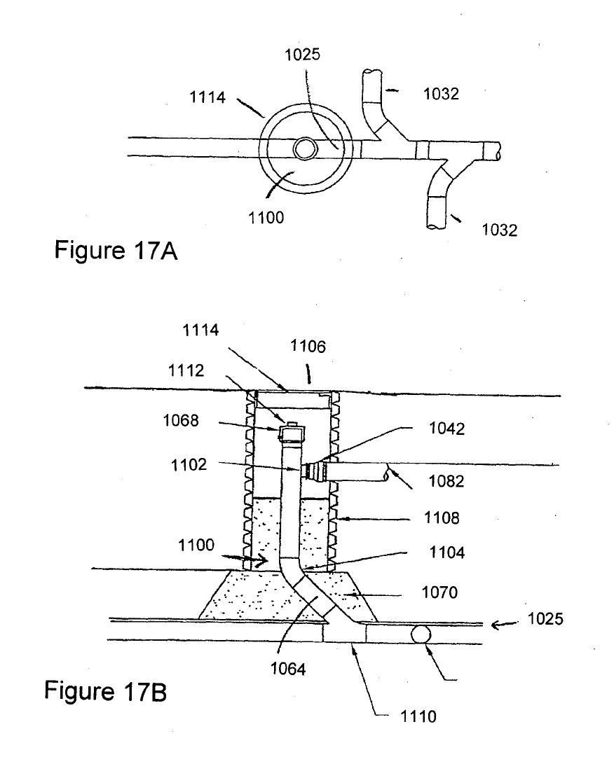

With reference to FIGS. 17A-B, there is provided a clean-out 1100 comprising a vertical stand pipe 1102 that is connected to a collection main by means of a joint 1110 and elbow joint 1104. The vertical pipe 1102 is sealed with a cap 1068 which comprises a vertical vent 1112.

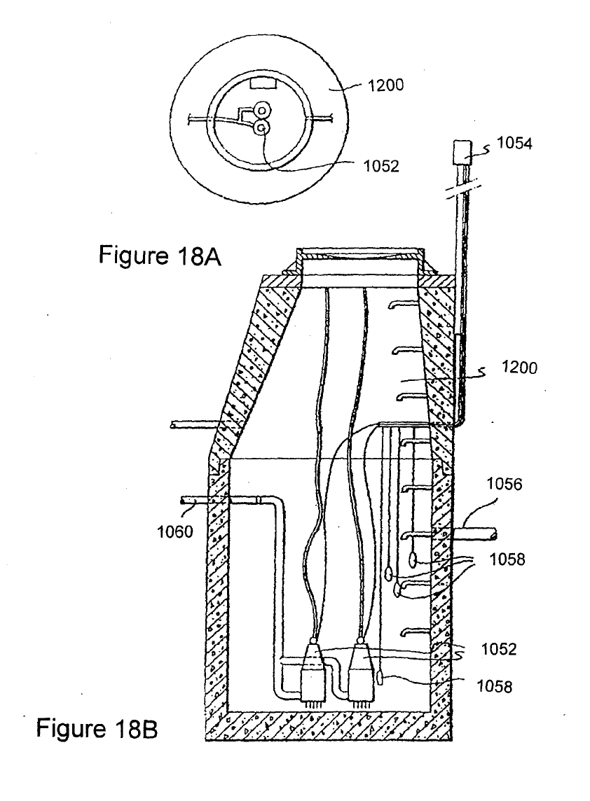

As detailed in FIGS. 18A-B, a pumping station 1200 is inserted into the sewage collection main to aid in the flow of the liquid effluent to the centralized sewage treatment center. A pumping station 1200 includes submersible pumps. 1052 wired to a control panel 1054 which is preferably located above ground. An inlet pipe 1056 from the collection main discharges liquid effluent into the, station 1200. The submersible pumps 1052 have a series of floats 1058 which activate pumps 1052 when the level of the liquid effluent in the pumping station 1100 reaches predetermined elevations. The liquid effluent is pumped out of the pumping station reservoir 1100 and into a force-main 1060 which carries the liquid effluent to the central sewage treatment center. Gaskets such as A-LOK gaskets are used to maintain airtight connections between the walls of the pumping station 200 and the inlet pipe 1056 and the force-main 1060. As only liquid effluent is pumped through the pumping station, the submersible pumps 1052 need only be liquid pumps rather than the typically more complex and expensive sewage pumps required in historic sewer systems.

The BGCs feature a larger volume first compartment than in historic septic tanks. Thus the first compartment can contain a larger sludge volume, which extends the cleanout cycle. The larger first compartment also acts as a surge suppressor to slow the flow of sewage through the system. Faster flow rates result in less settling, higher TSS levels and more sludge being conducted out of the BGC. HSS systems compensate for surges with redundant surge suppression tanks located throughout the system. Such tanks are not required in HPSS, resulting in reduction of cost and complexity of the system. Relatively large first compartments also allows longer hydraulic retention times, thus allowing more settling to occur before the sewage is conducted to the second compartment.

Decomposition of the sludge inside the BGC occurs as naturally occurring micro-organisms break down and digest the waste. Optimization of decomposition is desirable as it reduces or reverses the rate of accumulation of solids within the BGC which extends clean-out cycles. As mentioned earlier, the microbial digestion of sludge can be promoted by optimizing environmental conditions, such as temperature, pH, components, nutrient levels, moisture or water-content and aeration levels.

The BGC comprises means for optimizing one or more environmental conditions to promote microbial digestion. The impact of electrolysis of wastewaters and sludge accumulation can be assessed using a bench scale, study with the electrolysis anode and cathode probes being placed in the sludge layer. This will allow for the substantial optimization of both the process and the system.

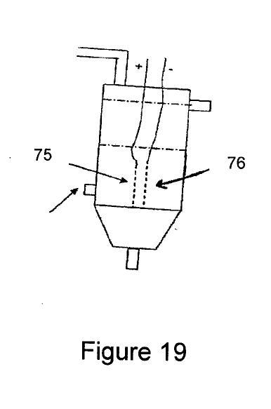

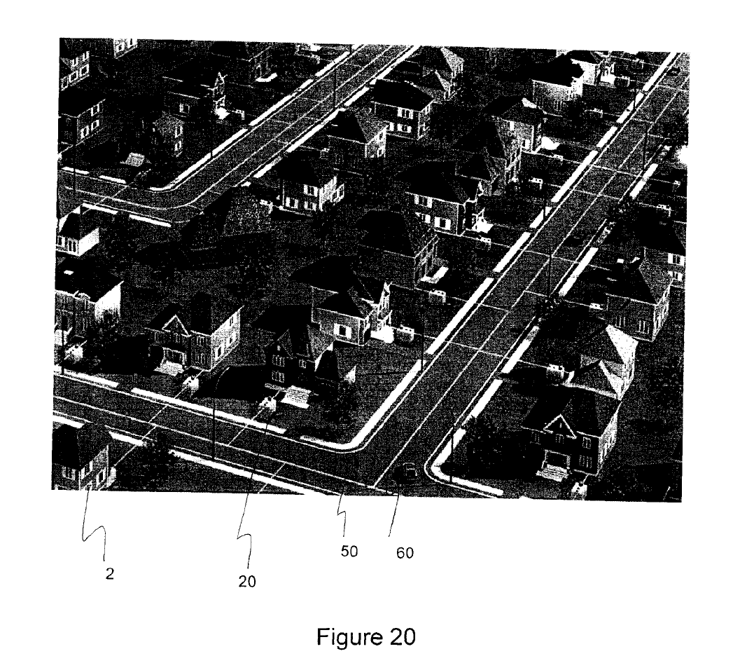

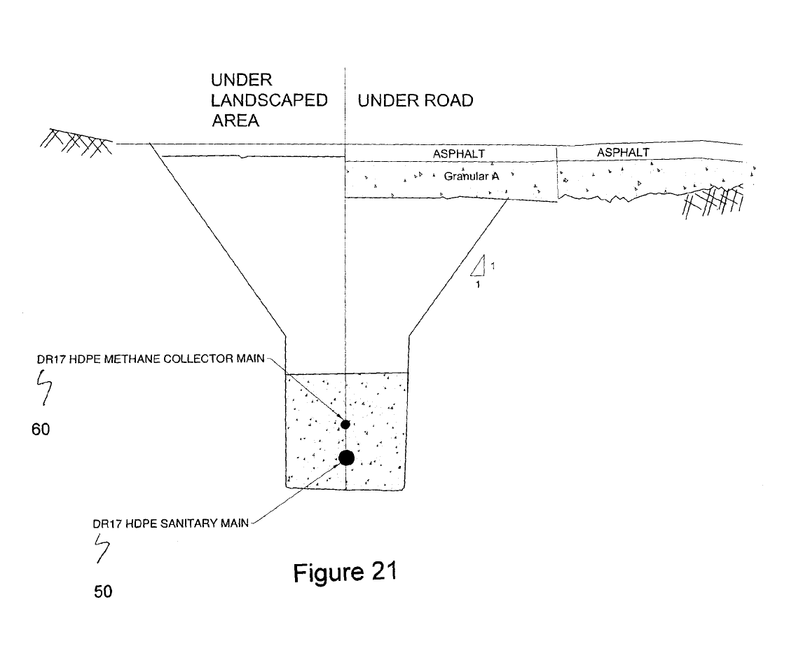

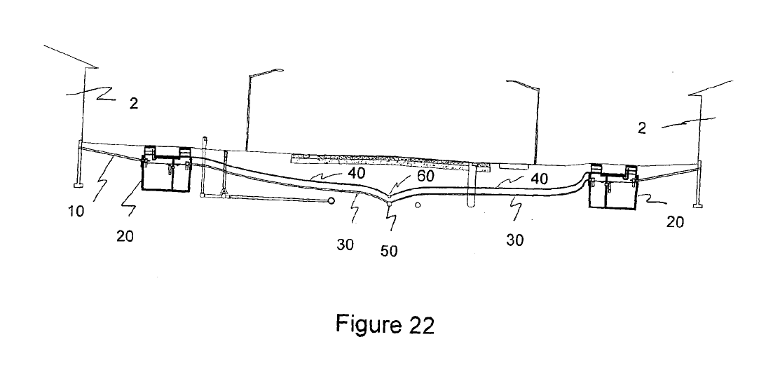

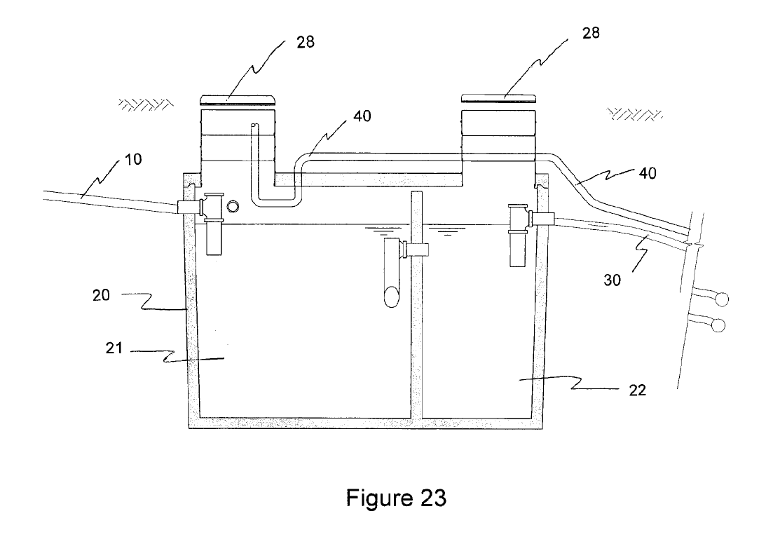

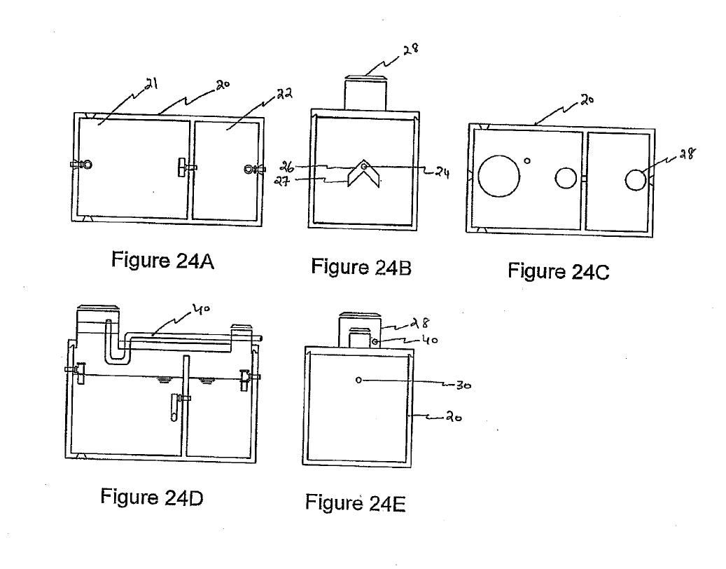

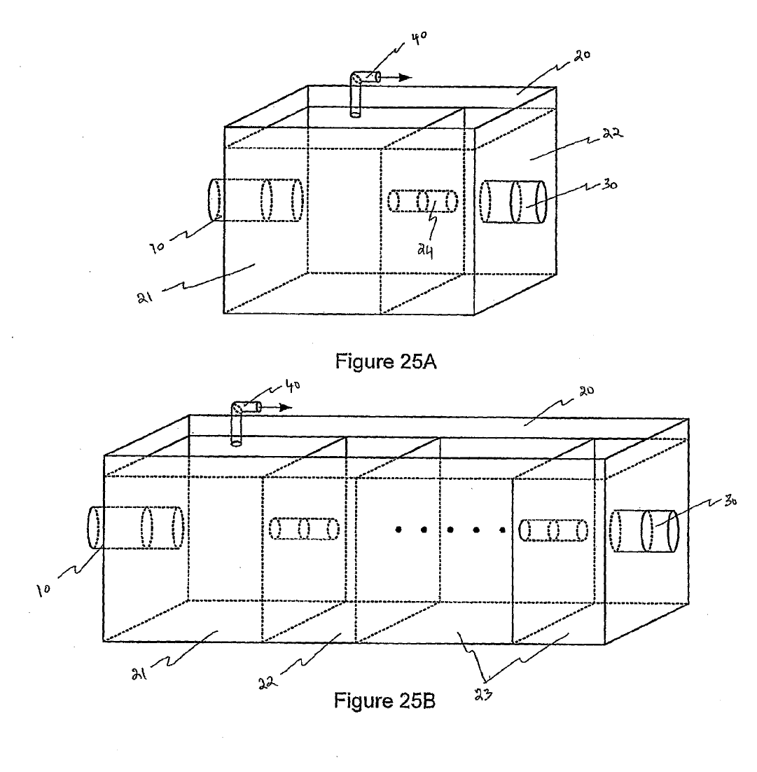

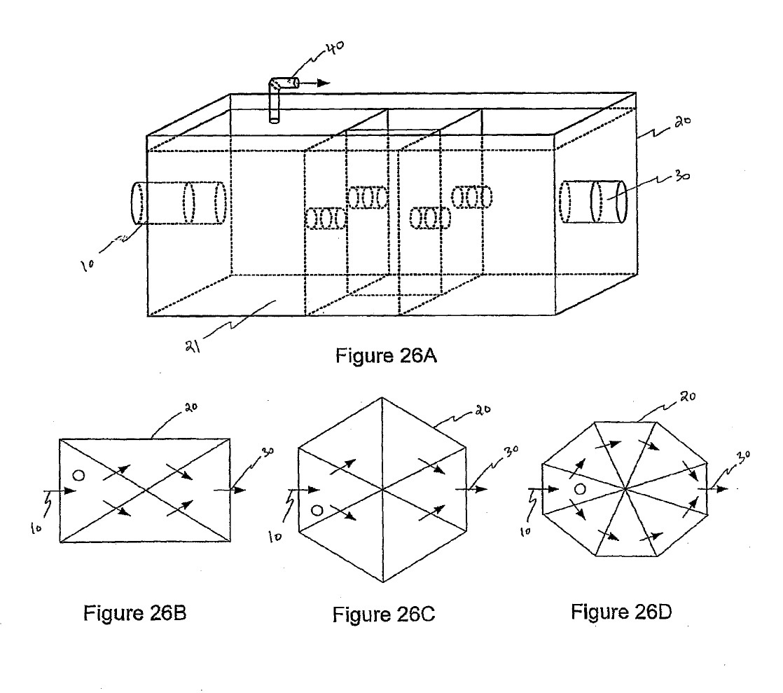

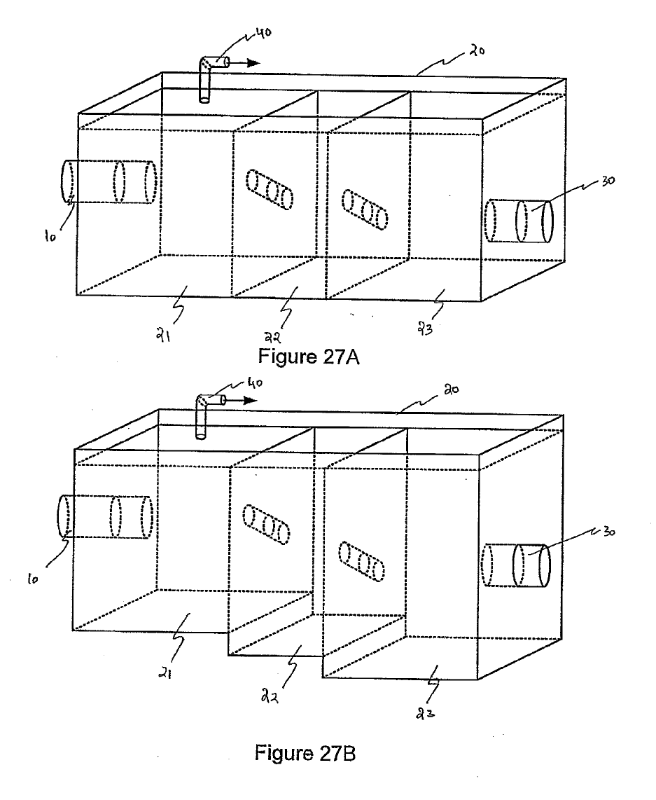

With reference to FIG. 19, the electrode assembly comprises a cathode 75 and an anode 76 separated by plastic spacers to a distance of approximately 5 mm. The cathode 75 can be a stainless steel cathode and the anode 76 can comprise a mixed metal oxide coated titanium mesh.

To determine optimal electrolysis conditions, either BGC sludge or septic tank sludge can be used and batch tests can be carried out to evaluate the electrochemical and microbiological mechanisms of sludge degradation allowing for optimization. As well, the overall efficiency of electrolysis can be evaluated in continuous or intermittent flow experiments. For comparisons, a blank (non-electrode) apparatus can also be tested.

Residential communities generally generate daily peak flows in the morning and early evening. All elements of any sewer system, including the centralized sewage treatment center, are designed for peak flows. In an HPSS, as the majority of solids remain within the individual BGCs, the peaking factor is substantially minimized. This eases the sizing constraints and ensures that the HPSS is a smaller, less complicated sewer system with lower capital costs to install and maintain that a traditional HSS.

The time period of peak flow rates can also be substantially shifted from traditional high demand periods. This ability to ‘peak shift’ provides additional capacity to an existing HSS limited by high volumes during peak demand periods, when retrofitted with these BGCs. Furthermore, the BGCs can reduce the amount of sludge that is treated in the centralized sewage treatment center.

Traditionally, HSS requires a fast flow rate to prevent build-up of sludge in the sewage collection mains. The HPSS allows for a low flow rate of liquid effluent due to the absence of sludge flowing through. The absence of sludge can also allow for easier cleaning of the system. This lower flow rate of the liquid effluent required by the HPSS can allow for more gentle gradients in the sewage collection main. Access points such as maintenance clean-outs and covers are provided along the system at spaced intervals, which are all sealingly connected to the system. Because of the substantial absence of solids in the liquid effluent and the ease of cleaning the system, these access points can be placed further apart than in an HSS. For the HPSS, flushing is typically required less often than in HSS and may occur approximately every seven to ten years, always after unit de-sludging.

It is obvious that the foregoing embodiments of the invention are exemplary and can be varied in many ways. Such present or future variations are not to be regarded as a departure from the spirit and scope of the invention, and all such modifications as would be obvious to one skilled in the art are intended to be included within the scope of the following claims.

1. A biogas capture and/or collection system comprising: one or more biogas capture and/or collection units configured so as to capture and/or collect substantially all of the biogas generated in one or more biogas generating chambers by the degradation of waste received from one or more sources of waste.

2. The biogas capture and/or collection system of claim 1 wherein the biogas capture and/or collection units are tubular conduits operatively linked to the biogas generating chambers.

3. The biogas capture and/or collection system of claim 1 wherein the biogas capture and/or collection units are containers (canisters) removably linked to the biogas generating chambers and designed for reversible capture of the biogas generated therein.

4. The biogas capture and/or collection system of claim 1 wherein the extracted biogas is utilized in gas utilization centers electricity production, use as fuels, and use for chemical synthesis.

5. The biogas capture and/or collection system of claim 4 wherein the gas utilization centers are located locally at the individual sources of waste.

6. The biogas capture and/or collection system of claim 4 wherein the gas utilization center is located at a centralized location.
